(19) United States
(12) Patent Application Publication
(10) Pub. No.: US 2019/0077868 A1
(43) Pub. Date: Mar. 14, 2019

(54) METHODS OF TREATING OR PREVENTING GRAFT VERSUS HOST DISEASE

(71) Applicant: Millennium Pharmaceuticals, Inc., Cambridge, MA (US)

(72) Inventors: Jessica A. SACHS, Needham, MA (US); John E. FORD, San Mateo, CA (US)

(21) Appl. No.: 16/084,392

(22) PCT Filed: Mar. 13, 2017
(86) PCT No.: PCT/US2017/022067

§ 371 Date: Sep. 12, 2018

Related U.S. Application Data
(60) Provisional application No. 62/307,896, filed on Mar. 14, 2016.
(60) Provisional application No. 62/420,825, filed on Nov. 11, 2016.

(51) Int. Cl. C07K 16/28; A61P 37/06; A61K 9/00

(52) U.S. Cl. CPC C07K 16/2839; A61P 37/06; C07K 2317/94; C07K 2317/24; C07K 2317/76; A61K 9/0019

(57) ABSTRACT

A method for treating or preventing GvHD in a human patient, comprising administering to a patient suffering from GvHD or at risk for GvHD, a humanized antibody having binding specificity for human α4β7 integrin.

### RELATED APPLICATIONS

This application claims the benefit of U.S. Provisional Application No. 62/307,896 filed on Mar. 14, 2016 and U.S. Provisional Application No. 62/420,825 filed on Nov. 11, 2016. The entire contents of the foregoing applications are hereby incorporated by reference.

### BACKGROUND

Allogeneic hematopoietic cell transplantation, such as hematopoietic stem cell transplantation (allo-HSCT) is an important therapy that is used to treat hematological malignant disorders and hematological genetic diseases, but its use is limited by the major complication of graft-versus-host disease (GvHD). GvHD following an allo-HSCT is a major cause of morbidity and mortality. The risk of GvHD is variable and depends on patient factors, donor factors, the degree of histocompatibility between donor and recipient, the conditioning regimen, and the GvHD prophylaxis strategy employed. Conditioning the patient for allo-HSCT permits engraftment of donor hematopoietic cells and involves chemotherapy or irradiation and is given immediately prior to a transplant. The purpose of conditioning is to help eradicate the patient's disease prior to the infusion of hematopoietic stem cells (HSC) and to suppress immune reactions. The post-transplant prognosis often includes acute and chronic graft-versus-host disease that may be life-threatening. In patients receiving allogeneic hematopoietic stem cells after myeloablative conditioning, the risk of Grade 2 to 4 acute GvHD is approximately 40% to 50%. The reduction of GvHD without causing significant systemic immunosuppression may improve overall outcomes following allo-HSCT.

GvHD results from an activation of alloreactive donor lymphocytes by histocompatibility antigens on host antigen-presenting cells (APCs). It has been postulated that intestinal microflora and endotoxin exert a crucial step in APC activation, and that this process occurs in the gut-associated lymphoid tissues (GALT). Clinically, GvHD can be reduced through the use of T-cell depletion strategies and gut decontamination, highlighting the respective roles of both T cells and gastrointestinal (GI) microflora on the development of GvHD. In clinical HSCT, expression of the human lymphocyte integrin α4β7 has been shown to be significantly increased on naïve and memory T cells in patients who subsequently developed intestinal acute GvHD compared with patients who developed skin acute GvHD or no GvHD. T-cell trafficking to GALT and the interaction between α4β7 and mucosal addressin cell adhesion molecule-1 (MAdCAM-1) has been studied in murine models of acute GvHD.

The risk of GvHD is variable and depends on patient factors, donor factors, the degree of histocompatibility between donor and recipient, the conditioning regimen, and the GvHD prophylaxis strategy. In patients receiving hematopoietic stem cells from an unrelated donor source after myeloablative conditioning, the risk of Grade 2, 3, or 4 acute GvHD is approximately 40% to 50%. Patients who develop acute GvHD have an increased risk of adverse events including infections related to immunosuppressive therapies for GvHD and the development of chronic GvHD. The combined mortality attributable to GvHD and infection is high in patients after allo-HSCT, second only to death due to primary disease. Additionally, the prognosis for patients who do not achieve a response after initial therapy for acute GvHD is poor.

GvHD prophylaxis is employed for all patients undergoing allo-HSCT using various strategies such as calcineurin inhibitors, methotrexate, and in vivo or ex vivo T-cell depletion; however, despite GvHD prophylaxis, GvHD still develops in 30% to 50% of allo-HSCT recipients (Gooley T A et al., N Engl J Med 2010; 363(22):2091-101; McDonald G B et al., Blood 2015; 126(1):113-20). First-line treatment for patients with acute GvHD (Grade II or higher) is corticosteroids such as methylprednisolone. Although first-line treatment is effective in more than 50% of patients, durable responses (defined as a complete response [CR] by Day 28 that remains at 6 months after onset) are observed in only one-third of patients (Levine J E et al., Lancet Haematol 2015; 2(1)e21-e9). In patients who do not respond to primary treatment with steroids, acute GvHD is associated with a high rate of morbidity and mortality, primarily from infections and/or multi-organ failure (Martinez C et al., Biol Blood Marrow Transplant 2009; 15(5):639-42; Xhaard A. et al., Biol Blood Marrow Transplant 2009; 15(5):639-42). Despite this, there are no approved or agreed-upon standard treatments for steroid-refractory GvHD, which remains largely an untreatable disease with limited survival, representing a major unmet therapeutic need.

Acute GvHD that occurs after allo-HSCT involves the skin, liver, and gut in the most severe and life-threatening cases. Acute skin GvHD is generally not life-threatening with existing therapies, which are usually effective, and the incidence of Stage 3 or 4 liver GvHD is around 2% (Gooley T A et al., N Engl J Med 2010; 363(22):2091-101). While the incidence of Stage 3 or 4 intestinal GvHD has decreased in recent years, most courses of treatment remain unsuccessful, with most fatal cases of GvHD involving the gastrointestinal (GI) tract (Gooley T A et al., N Engl J Med 2010; 363(22):2091-101). Lower intestinal GvHD presents with secretory, protein-rich diarrhea (in excess of 1.5 liters per day in severe cases), abdominal pain from gut distention, inflammation of the small intestine and colon, mucosal ulceration, and bleeding. A study of patients who received allo-HSCT showed that 7.9% of patients developed Stage 3 or 4 intestinal GvHD at a median time to onset of 35 days after transplant (Castilla-Llorente C et al., Bone Marrow Transplant 2014; 49(7):966-71). Of these patients, 73% developed corticosteroid resistance before or within 14 days of onset of Stage 3 or 4 intestinal GvHD. Significant risk factors for mortality include corticosteroid resistance, age >18 years, increased serum bilirubin, and overt GI bleeding. Thus, there remains an urgent unmet medical need for agents and methods to treat or prevent of acute GvHD.

### SUMMARY OF THE INVENTION

The invention relates to methods of treating or preventing graft versus host disease, by administering an antagonist of human α4β7 integrin to a subject in need thereof. The invention relates to the prevention of graft versus host disease (GvHD) with an antagonist of the α4β7 integrin, such as an anti-α4β7 antibody, such as a humanized anti-α4β7 antibody (e.g., vedolizumab). In some embodiments, the patient has acute lymphoblastic leukemia (ALL) or acute myeloid leukemia (AML).

GvHD is a major cause of morbidity and mortality in patients undergoing allo-HSCT. The significant mortality from GvHD limits the use of HSCT as a potentially curative therapy for disease, e.g., malignant disease. Reducing nonrelapse mortality (such as from GvHD and infection) may improve overall survival after allo-HSCT. Steroids and other systemic immunosuppressive agents (such as tacrolimus+short-term methotrexate) are the current standard of care (SOC) used to prevent and treat GvHD. However, this standard of care can increase the risk of infections, and is also not completely effective. Immunosuppression geared at reducing GvHD can also decrease graft-versus-tumor (GvT) effects. Therefore, reducing GvHD without systemic immunosuppression, as described in the present invention, has the potential to improve overall outcomes in allo-HSCT and possibly extend and/or save lives from this disease.

Following allo-HSCT, naïve T cells in the hematopoietic stem cells (HSC) inoculum expressing low levels of α4β7 integrin circulate to host Peyer's patches (PP), or mesenteric lymph nodes (MLN), where they encounter intestinal microbial antigens in the context of alloantigens and are activated. These activated effector T cells upregulate α4β7 integrin, next home toward the intestinal mucosa via the α4β7/MADCAM-1 pathway, and generate intestinal mucosal damage. The interaction between alloreactive effector T cells, intestinal microbes, and intestinal mucosal tissues leads to release of numerous inflammatory mediators, creating a positive feedback loop. The combination of expansion of alloreactive T cells, breakdown of intestinal barriers leading to translocation of microbes and microbial stimuli, and a systemic cytokine storm lead to diffuse systemic symptoms of GvHD.

For the prevention of GvHD, without wishing to be bound by any particular theory, it is believed that the present invention blocks the initial trafficking of T cells to secondary lymphoid organs, e.g., PP or MLN, by interfering with the α4β7/MADCAM-1 pathway. Thus, the present invention suppresses and/or prevents the evolution of acute GvHD. In some embodiments, the present invention provides for a 50% reduction in cumulative incidence & severity of acute GVHD at Day 100 and 25% reduction in 1 year mortality as compared to the current standard of care (SOC). In another embodiment, the present invention improves GvHD-free survival at 6 months and improves GvHD-free and relapse-free survival at 1 year; improved cumulative incidence and severity of acute GvHD at 6 months following HSCT; improved cumulative incidence of chronic GVHD requiring immunosuppression at 12 months; or improved GRFS (GvHD-free and relapse-free survival) compared to SOC. In some embodiments, administration of an α4β7 integrin antagonist, such as an anti-α4β7 antibody, results in a 5%, 10%, 15%, 20%, 25%, 30% reduction in the risk of mortality, e.g., from 40% to e.g., 35% or 30% or less risk of mortality from acute GvHD.

In one aspect, the invention relates to a method of preventing graft versus host disease (GvHD), wherein the method comprises the step of: administering to a human patient undergoing allogeneic hematopoietic stem cell transplantation (allo-HSCT), a humanized antibody having binding specificity for human α4β7 integrin, wherein the humanized antibody is administered to the patient according to the following dosing regimen:

a. an initial dose of 75 mg, 300 mg, 450 mg or 600 mg of the humanized antibody as an intravenous infusion the day before allo-HSCT;

b. followed by a second subsequent dose of 75 mg, 300 mg, 450 mg or 600 mg of the humanized antibody as an intravenous infusion at about two weeks after the initial dose;

c. followed by a third subsequent dose of 75 mg, 300 mg, 450 mg or 600 mg of the humanized antibody as an intravenous infusion at about six weeks after the initial dose; wherein the dosing regimen results in Grade II GvHD, Grade I GvHD or no GvHD, further wherein the humanized antibody comprises an antigen binding region of nonhuman origin and at least a portion of an antibody of human origin, wherein the humanized antibody has binding specificity for the α4β7 complex, wherein the antigen-binding region comprises the Light chain CDRs of SEQ ID NO:7 (CDR1), SEQ ID NO:8 (CDR2) and SEQ ID NO:9 (CDR3); and Heavy chain CDRs: SEQ ID NO:4 (CDR1), SEQ ID NO:5 (CDR2) and SEQ ID NO:6 (CDR3).

In another aspect, the invention relates to a method of reducing the occurrence of acute graft versus host disease (GvHD), wherein the method comprises the step of: administering to a human patient undergoing allogeneic hematopoietic stem cell transplantation (allo-HSCT), a humanized antibody having binding specificity for human α4β7 integrin, wherein the humanized antibody is administered to the patient according to the following dosing regimen:

a. an initial dose of 75 mg, 300 mg, 450 mg or 600 mg of the humanized antibody as an intravenous infusion the day before allo-HSCT;

b. followed by a second subsequent dose of 300 mg of the humanized antibody as an intravenous infusion at about two weeks after the initial dose;

c. followed by a third subsequent dose of 300 mg of the humanized antibody as an intravenous infusion at about six weeks after the initial dose; wherein the humanized antibody comprises an antigen binding region of nonhuman origin and at least a portion of an antibody of human origin, wherein the humanized antibody has binding specificity for the α4β7 complex, wherein the antigen-binding region comprises the Light chain CDRs of SEQ ID NO:7 (CDR1), SEQ ID NO:8 (CDR2) and SEQ ID NO:9 (CDR3); and Heavy chain CDRs: SEQ ID NO:4 (CDR1), SEQ ID NO:5 (CDR2) and SEQ ID NO:6 (CDR3).

In another aspect, the invention relates to a method of reducing the severity of acute graft versus host disease (GvHD), wherein the method comprises the step of: administering to a human patient undergoing allogeneic hematopoietic stem cell transplantation (allo-HSCT), a humanized antibody having binding specificity for human α4β7 integrin, wherein the humanized antibody is administered to the patient according to the following dosing regimen:

a. an initial dose of 300 mg, 450 mg or 600 mg of the humanized antibody as an intravenous infusion the day before allo-HSCT;

b. followed by a second subsequent dose of 300 mg of the humanized antibody as an intravenous infusion at about two weeks after the initial dose;

c. followed by a third subsequent dose of 300 mg of the humanized antibody as an intravenous infusion at about six weeks after the initial dose; wherein the humanized antibody comprises an antigen binding region of nonhuman origin and at least a portion of an antibody of human origin, wherein the humanized antibody has binding specificity for the α4β7 complex, wherein the antigen-binding region comprises the Light chain CDRs of SEQ ID NO:7 (CDR1), SEQ ID NO:8 (CDR2) and SEQ ID NO:9 (CDR3); and Heavy chain CDRs: SEQ ID NO:4 (CDR1), SEQ ID NO:5 (CDR2) and SEQ ID NO:6 (CDR3).

In some embodiments, reducing the severity of acute graft versus host disease (GvHD) results in Grade I or Grade II GvHD, per modified Glucksberg criteria, or similar severity of GvHD per other scoring system, or no GvHD. In some embodiments, reducing the severity of acute GvHD is a 50% reduction in cumulative incidence and severity of Grade II-IV or Grade III-IV acute GvHD at Day 100 as compared to treatment with methotrexate and calcineurin inhibitor alone. In some embodiments, reducing the severity of acute graft versus host disease (GvHD) is a reduction in 1 year mortality as compared to treatment with methotrexate and calcineurin inhibitor alone.

In some embodiments, the patient is identified as at risk of acute GvHD after measurement of criteria selected from the group consisting of biomarkers, clinical signs and refractoriness to steroid use.

In some embodiments, the humanized antibody is administered more than 15 days, more than 16 days, more than 17 days, more than 20 days, or more than 21 days after hematopoietic stem cell infusion.

In some embodiments, reducing the occurrence of acute GvHD results in Grade I or Grade II GvHD, per modified Glucksberg criteria, or similar severity of GvHD per other scoring system, or no GvHD. In other embodiments, reducing the occurrence of acute GvHD is a 50% reduction in cumulative incidence and severity of Grade II-IV or Grade III-IV acute GvHD at Day 100 as compared to treatment with methotrexate and calcineurin inhibitor alone. In other embodiments, reducing the occurrence of acute graft versus host disease (GvHD) is a reduction in 1 year mortality as compared to treatment with methotrexate and calcineurin inhibitor alone.

In another aspect, the invention relates to a method of treating a patient suffering from cancer or a nonmalignant hematological, immunological disease or autoimmune disease, comprising the steps of

a. conditioning the immune system of the patient for hematopoietic stem cell transplant,

b. administering a humanized antibody having binding specificity for human α4β7 integrin,

c. waiting at least 12 hours,

d. administering allogeneic hematopoietic stem cells,

e. waiting thirteen days, then administering a second dose of humanized antibody having binding specificity for human α4β7 integrin, and

f. waiting four weeks, then administering a third dose of humanized antibody having binding specificity for human α4β7 integrin.

In another aspect, the invention relates to a method of suppressing an immune response in a cancer patient, wherein the method comprises the step of: administering to a human patient undergoing allogeneic hematopoietic stem cell transplantation (allo-HSCT), a humanized antibody having binding specificity for human α4β7 integrin, wherein the humanized antibody is administered to the patient according to the following dosing regimen:

a. an initial dose of 75 mg, 300 mg, 450 mg or 600 mg of the humanized antibody as an intravenous infusion the day before allo-HSCT;

b. followed by a second subsequent dose of 300 mg of the humanized antibody as an intravenous infusion at about two weeks after the initial dose;

c. followed by a third subsequent dose of 300 mg of the humanized antibody as an intravenous infusion at about six weeks after the initial dose; further wherein the humanized antibody comprises an antigen binding region of nonhuman origin and at least a portion of an antibody of human origin, wherein the humanized antibody has binding specificity for the α4β7 complex, wherein the antigen-binding region comprises the Light chain CDRs of SEQ ID NO:7 (CDR1), SEQ ID NO:8 (CDR2) and SEQ ID NO:9 (CDR3); and Heavy chain CDRs: SEQ ID NO:4 (CDR1), SEQ ID NO:5 (CDR2) and SEQ ID NO:6 (CDR3).

The humanized antibody may have a heavy chain variable region sequence of amino acids 20 to 140 of SEQ ID NO:1.

The humanized antibody may have a light chain variable region sequence of amino acids 20 to 131 of SEQ ID NO:2.

The humanized antibody may have a heavy chain comprising amino acids 20 to 470 of SEQ ID NO:1 and a light chain comprising amino acids 20 to 238 of SEQ ID NO:2. In some embodiments, the humanized antibody is vedolizumab.

In a further aspect, the invention relates to a method of treating a transplant patient, wherein the transplant patient is a recipient of an infusion of allogeneic hematopoietic cells, comprising administering an anti-α4β7 antagonist. In some embodiments, the α4β7 integrin antagonist is an anti-α4β7 antibody. In some embodiments, the anti-α4β7 antibody is a humanized antibody. In some embodiments, the anti-α4β7 antagonist is administered as a single dose 10 to 28 days, 14 to 30 days, 15 to 32 days, or 15 to 35 days after the infusion.

In additional aspects, the disclosure provides a method for treating graft versus host disease (GvHD) in a human, comprising administering to a human in need thereof an antibody that has binding specificity for the human α4β7 integrin complex. In one example, the antibody that has binding specificity for the human α4β7 integrin complex is administered according to the following regimen: a) a first dose of antibody; b) a second dose of antibody about two weeks after the first dose; c) a third dose of antibody about four weeks after the second dose; and optionally d) further doses of antibody, wherein each further dose is administered about four weeks after the immediate prior dose; and wherein each dose in a)-d) is 300 mg, or each dose in a)-d) is 600 mg. In some embodiments, a patient who receives five doses in a)-d) at 300 or 600 mg antibody in each dose may further repeat a)-d) at antibody doses of 300 mg each dose.

### BRIEF DESCRIPTION OF THE DRAWINGS

FIG. 1 is a schematic illustrating an overview of the study design from days −1 to +50. Allo-HSCT occurs on day 0. Vedolizumab is administered the day before the allo-HSCT (day −1), and on days +13 and +42 after allo-HSCT.

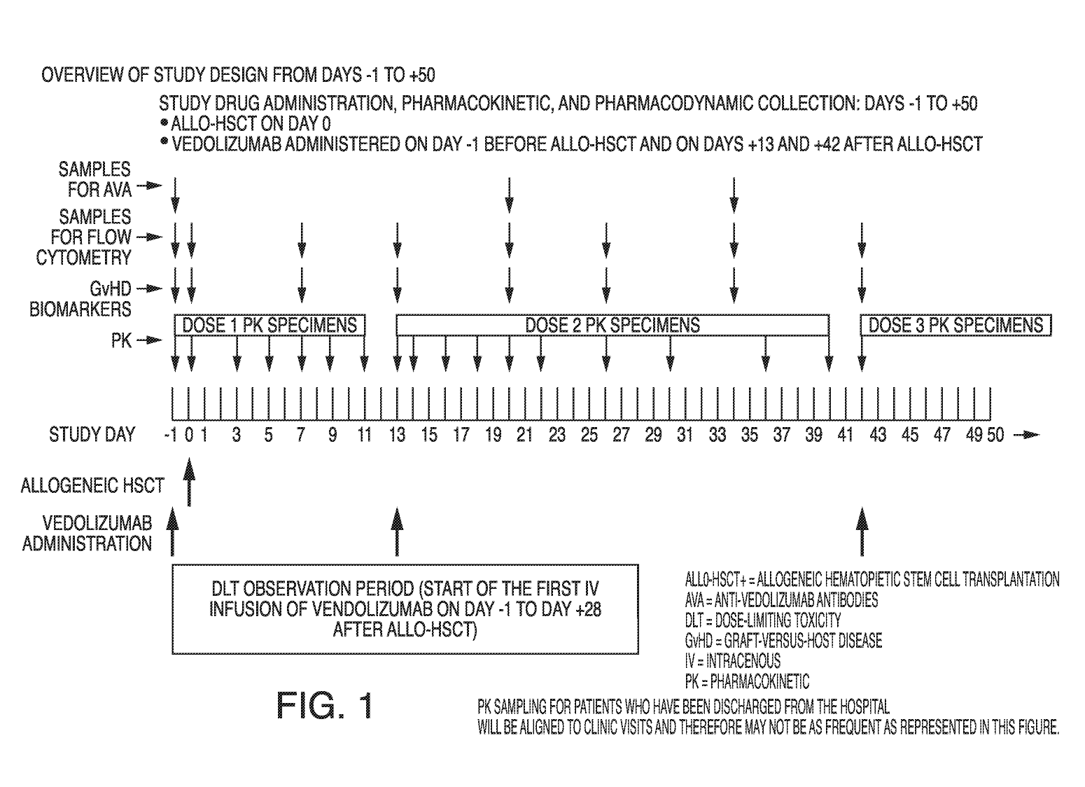

FIG. 2 illustrates how blocking the α4β7/MADCAM-1 interaction in GALT and MLNs may reduce the generation of allo-reactive memory T cells and their subsequent entry into the gut, thereby reducing the occurrence of GvHD.

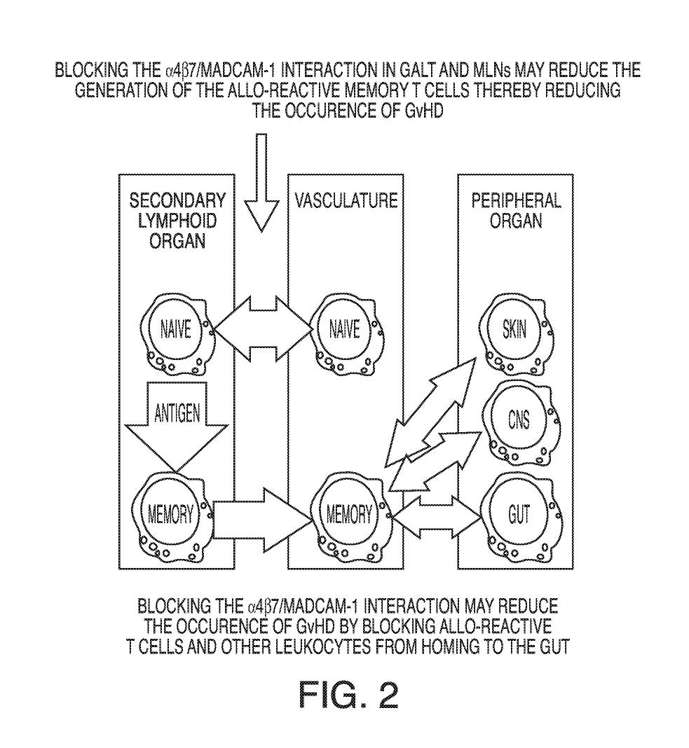

FIG. 3 is a graph showing simulated and observed PK data from three patients. The PK simulated data is shown by the region between the jagged lines (2.5 and 97.5 percentiles of simulated data), the dashed black line without dots represents the median of simulated data, the points and lines are individual observed data plotted using nominal times, and the horizontal dashed line represents the LLOQ of 0.2 mcg/mL.

### DETAILED DESCRIPTION

The present invention relates to a method of treating disease through preventing GvHD. The method comprises administering an α4β7 integrin antagonist, such as an anti-α4β7 antibody, to a patient undergoing allogeneic hematopoietic cell transplant, such as allogeneic hematopoietic stem cell transplant (allo-HSCT). In some embodiments, the disease suffered by the patient is cancer, e.g., hematological cancer (such as leukemia, lymphoma, myeloma or myelodysplastic syndrome). In other embodiments, the disease suffered by the patient is characterized by a nonmalignant hematological or immunological defect (such as a bone marrow failure syndrome, hemoglobinopathy, or SCID). In one aspect, the transplant patient is conditioned, e.g., undergoes a process to prepare the body to receive the transplant. In some embodiments, the conditioning is myeloablative conditioning (“myelo conditioning”) or reduced-intensity conditioning (RIC), e.g., less, such as 10%, 20%, 30%, 40%, 20-40%, 30-50% or 50% less, of the agents used in myeloablative conditioning. In some embodiments, the conditioning is chemically-induced, e.g. by cyclophosphamide and/or busulfan and/or fludarabine, radiation-induced, e.g., by total body irradiation, or induced by a combination of chemical treatment and radiation, such as cyclophosphamide and total body irradiation.

In one aspect, the patient, e.g., transplant patient, is administered allogeneic hematopoietic cells, e.g., as an infusion. In some embodiments, the allogeneic hematopoietic cells are allogeneic hematopoietic stem cells, i.e., the patient receives an allogeneic hematopoietic stem cell transplant (allo-HSCT). In some embodiments, the allogeneic hematopoietic cells are allogeneic leukocytic cells. In some embodiments, the allogeneic leukocytic cells comprise lymphocytes, e.g., T-lymphocytes. In some embodiments, the allogeneic leukocytic cells comprise lymphocytes expressing a chimeric antigen receptor. In some embodiments, the allogeneic leukocytic cells comprise natural killer cells. In some embodiments, the allogeneic leukocytic cells comprise cytotoxic T-lymphocytes, e.g., T-cells expressing CD8. In some embodiments, the allogeneic leukocytic cells are selected to consist of at least 30%, 40%, 50%, 60%, 70%, 80%, 85%, 90%, 95%, 96%, 97%, 98%, 99% or 100% lymphocytes. In some embodiments, the allogeneic leukocytic cells are selected to consist of at least 30%, 40%, 50%, 60%, 70%, 80%, 85%, 90%, 95%, 96%, 97%, 98%, 99% or 100% T-lymphocytes. In some embodiments, the allogeneic hematopoietic cells have one or more recombinant modifications known in the art to control their behavior in the patient.

In some embodiments, the α4β7 antagonist, such as an anti-α4β7 antibody, prevents graft versus host disease (GVHD). In some embodiments, the α4β7 antagonist, such as an anti-α4β7 antibody, does not prevent graft versus tumor activity. In some embodiments, the transplanted cells engraft with tolerance to the patient's tissues. In some embodiments, the invention relates to methods of preventing graft versus host disease (GvHD) by administering an anti-α4β7 antibody to a patient undergoing allo-HSCT. In some embodiments, the α4β7 antagonist is administered to a patient prior to receiving hematopoietic cells, such as allogeneic hematopoietic stem cells, and further is provided during hematopoietic cell engraftment, and thereby prevents GVHD. In other embodiments, the α4β7 antagonist is administered to a patient shortly after, such as up to seven days after, receiving the hematopoietic cells. In some embodiments, the anti-α4β7 antibody is a humanized antibody, e.g., a humanized antibody with the epitopic specificity of Act-1 mouse monoclonal antibody. In some embodiments, the anti-α4β7 antibody is vedolizumab.

The hematopoietic cells, e.g., stem cells, may be derived from bone marrow or from blood (e.g., peripheral blood or umbilical cord blood) of a non-self donor, i.e., allogeneic. In some embodiments, the hematopoietic cells, e.g., stem cells, may be manipulated before infusion, e.g., enriched for or depleted of certain cells by antibody-selection or other mechanism, expanded in vitro, or subjected to gene editing or gene therapy. Examples of compositions of hematopoietic cells which are enriched or depleted for infusion include cells, which can be collected by e.g., negative selection, e.g., separation of leukocytes from red blood cells (e.g., differential centrifugation through a dense sugar or polymer solution (e.g., FICOLL® solution (Amersham Biosciences division of GE healthcare, Piscataway, N.J.) or HISTOPAQUE®-1077 solution, Sigma-Aldrich Biotechnology LP and Sigma-Aldrich Co., St. Louis, Mo.)) and/or positive selection by binding cells to a selection agent (e.g., a reagent which binds to a B-cell marker, such as CD19 or CD20, a myeloid progenitor marker, such as CD34, CD38, CD117, CD138, CD133, or ZAP70, or to a T-cell marker, such as CD2, CD3, CD4, CD5 or CD8 for direct isolation (e.g., the application of a magnetic field to solutions of cells comprising magnetic beads (e.g., from Miltenyi Biotec, Auburn, Calif.) or other beads, e.g., in a column (R&D Systems, Minneapolis, Minn.) which bind to the cell markers) or fluorescent-activated cell sorting). In one embodiment, the differential centrifugation concentrates a cell layer comprising leukocytes.

In some embodiments, the patient is suffering from a disease, such as cancer or a non-malignant disease. In some embodiments, the patient has leukemia, for example, acute lymphoblastic leukemia (ALL) or acute myeloid leukemia (AML). In some embodiments, the patient has a myelodysplastic or myeloproliferative disease. In some embodiments, the patient has lymphoma, such as non-Hodgkin's lymphoma or Hodgkin's lymphoma. In some embodiments, the patient has a nonmalignant hematological disorder, such as a hemoglobinopathy, e.g., sickle cell disease or thalassemia, bone marrow failure syndrome, e.g., aplastic anemia, Fanconi's anemia, or other marrow failure syndromes, an immune disease, such as severe combined immunodeficiency (SCID) or autoimmune disease, such as diabetes. In some embodiments, the patient has a disorder treatable with an organ transplant, such as sclerosing cholangitis, cirrhosis, or hemochromatosis (e.g., for a liver transplant); congestive heart disease, dilated coronary myopathy, or severe coronary artery disease (e.g., for a heart transplant); cystic fibrosis, chronic obstructive pulmonary disease, or pulmonary fibrosis (e.g., for a lung transplant); or diabetes, polycystic kidney disease, systemic lupus erythamatosus, or focal segmental glomerulosclerosis (e.g., for a kidney transplant). In some embodiments, the patient is receiving two transplants, for example a hematopoietic cell transplant, e.g., for the purpose of tolerance induction, and a solid organ transplant, e.g., transplant of a liver, a heart, a lung or a kidney. In another example, the patient is receiving two transplants, first an allo-HSCT and second, allogeneic T cells via donor leukocyte infusion (DLI). In this example, there is potential for development of acute GvHD in both transplant procedures and thus administration of an α4β7 integrin antagonist, such as an anti-α4β7 antibody, to a patient may be useful for both transplants.

Acute graft-versus-host-disease is characterized by damage to tissues such as the liver, skin (rash), gastrointestinal tract, and other mucosa caused by alloreactive immune cells such as T-cells. In some embodiments, autoreactive immune cells, may cause acute graft-versus-host disease Immune cells may become reactive from the hematopoietic cell infusion, or activated upon recognition of signals in tissues of the patient, e.g., the transplant patient, Signals recognized by alloreactive hematopoietic cells or autoreactive immune cells may be induced from the conditioning regimen or from tumor lysis syndrome, e.g., as a result of GVT activity. Prevention of GvHD may result from sustained α4β7 blockade beginning at the time of hematopoietic cell, e.g., hematopoietic stem cell infusion. Prophylactic administration of vedolizumab to patients undergoing allo-HSCT may prevent trafficking of alloreactive T-cells to GALT, (e.g., Peyer's patches) or mesenteric lymph nodes, and GI mucosa, thereby preventing the development of acute GvHD. Sustained α4β7 blockade may further prevent GvHD during hematopoietic cell engraftment, e.g., to block autoreactive immune cells. The anti-α4β7 antibody is provided at a dose sufficient to achieve sustained receptor saturation throughout the first 100 days following allo-HSCT, the time period in which the vast majority of acute GvHD occurs. Grade III-IV or index C-D acute GvHD is a risk factor for the development of chronic GvHD, so therapies that can prevent acute GvHD may reduce the risk of the development of chronic GvHD (Flowers M. E. D. et al. Blood 2011 Mar. 17 117(11):3214-19).

One aspect of the invention relates to an α4β7 integrin antagonist (e.g., vedolizumab) for use in the prevention of GvHD. Unlike healthy subjects, patients undergoing a conditioning regimen, e.g., myeloablative or reduced intensity conditioning, followed by hematopoietic cell transplant, such as allo-HSCT are expected to have markedly changing T-cell populations with variable α4β7 integrin expression during the post-transplant period. For example, engraftment of HSCs comprises homing of the engrafting HSCs to the bone marrow and maturation and homing of donor lymphocytes to secondary lymphoid organs and other tissues causing high susceptibility of the patient to infection while the engraftment occurs. Systemic treatments, e.g., administration of immunosuppressive agents (such as corticosteroids, cyclosporine, methotrexate and mycophenolate mofetil, and antibody therapies like alemtuzumab, anti-thymocyte globulin, or rituximab, and anti-TNF therapies) used to control aberrant activation of lymphocytes may affect the engraftment and the response to the graft or disease, e.g., cancer or nonmalignant hematological disorder. Gut selective therapies (such as anti-α4β7 antibody) offer the potential to decrease the generation and homing of allo-reactive gut specific lymphocytes in this setting while potentially preserving the GVT effect of the graft.

Another aspect of the invention relates to an α4β7 integrin antagonist (e.g., vedolizumab) for use in treating GvHD, such as steroid refractory acute intestinal GvHD, and methods of treating GvHD, such as steroid refractory acute intestinal GvHD, by administering an α4β7 integrin antagonist (e.g., vedolizumab) to a subject in need thereof.

### Definitions

The term “pharmaceutical formulation” refers to a preparation that contains an α4β7 antagonist, such as an anti-α4β7 antibody, in such form as to permit the biological activity of the antibody to be effective, and which contains no additional components which are unacceptably toxic to a subject to which the formulation would be administered.

The cell surface molecule, “α4β7 integrin,” or “α4β7,” is a heterodimer of an α₄ chain (CD49D, ITGA4) and a β₇ chain (ITGB7). Each chain can form a heterodimer with an alternative integrin chain, to form α₄β₁ or α_{E}β₇. Human α₄ and β₇ genes (GenBank (National Center for Biotechnology Information, Bethesda, Md.) RefSeq Accession numbers NM_000885 and NM_000889, respectively) are expressed by B and T lymphocytes, particularly memory CD4+ lymphocytes. Typical of many integrins, α4β7 can exist in either a resting or activated state. Ligands for α4β7 include vascular cell adhesion molecule (VCAM), fibronectin and mucosal addressin (MAdCAM (e.g., MAdCAM-1)).

An “α4β7 antagonist” is a molecule which antagonizes, reduces or inhibits the function of α4β7 integrin. Such antagonist may antagonize the interaction of α4β7 integrin with one or more of its ligands. An α4β7 antagonist may bind either chain of the heterodimer or a complex requiring both chains of the α4β7 integrin, or it may bind a ligand, such as MAdCAM. An α4β7 antagonist may be an antibody which performs such binding function, such as an anti-α4β7-integrin antibody or “anti-α4β7 antibody”. In some embodiments, an α4β7 antagonist, such as an anti-α4β7 antibody, has “binding specificity for the α4β7 complex” and binds to α4β7, but not to α4β1 or αEβ7.

The term “antibody” or “antibodies” herein is used in the broadest sense and specifically covers full length antibody, antibody peptide(s) or immunoglobulin(s), monoclonal antibodies, chimeric antibodies (including primatized antibodies), polyclonal antibodies, human antibodies, humanized antibodies and antibodies from non-human species, including human antibodies derived from a human germline immunoglobulin sequence transduced into the non-human species, e.g., mouse, sheep, chicken or goat, recombinant antigen binding forms such as monobodies and diabodies, multispecific antibodies (e.g. bispecific antibodies) formed from at least two full length antibodies (e.g., each portion comprising the antigen binding region of an antibody to a different antigen or epitope), and individual antigen binding fragments of any of the foregoing, e.g., of an antibody or the antibody from which it is derived, including dAbs, Fv, scFv, Fab, F(ab)′₂, Fab′.

The term “monoclonal antibody” as used herein refers to an antibody obtained from a population of substantially homogeneous antibodies, i.e., the individual antibodies comprising the population are identical and/or bind the same epitope. The modifier “monoclonal” indicates the character of the antibody as being obtained from a substantially homogeneous population of antibodies, and is not to be construed as requiring production of the antibody by any particular method.

“Antigen binding fragments” of an antibody preferably comprise at least the variable regions of the heavy and/or light chains of an anti-α4β7 antibody. For example, an antigen binding fragment of vedolizumab can comprise amino acid residues 20-131 of the humanized light chain sequence of SEQ ID NO:2 and amino acid residues 20-140 of the humanized heavy chain sequence of SEQ ID NO:1. Examples of such antigen binding fragments include Fab fragments, Fab′ fragments, Fv fragments, scFv and F(ab′)₂ fragments. Antigen binding fragments of an antibody can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a recombinant construct encoding the heavy chain of an F(ab′)₂ fragment can be designed to include DNA sequences encoding the CH_{I} domain and hinge region of the heavy chain. In one aspect, antigen binding fragments inhibit binding of α4β7 integrin to one or more of its ligands (e.g. the mucosal addressin MAdCAM (e.g., MAdCAM-1), fibronectin).

A “therapeutic monoclonal antibody” is an antibody used for therapy of a human subject. Therapeutic monoclonal antibodies disclosed herein include anti-α4β7 antibodies. Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), and the like. To assess ADCC activity of a molecule of interest, an in vitro ADCC assay, such as those described in U.S. Pat. No. 5,500,362 or 5,821,337 may be performed.

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of antibodies are well known.

The “light chains” of antibodies from any vertebrate species can be assigned to one of two clearly distinct types, called kappa (κ) and lambda (λ), based on the amino acid sequences of their constant domains.

The term “hypervariable region” when used herein refers to the amino acid residues of an antibody which are responsible for antigen binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

An “affinity matured” antibody has one or more alterations in one or more hypervariable regions thereof which result an improvement in the affinity of the antibody for antigen, compared to a parent antibody which does not possess those alteration(s). In one aspect, affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992). An “isolated” antibody is one which has been identified and separated and/or recovered from a component of its natural environment. In certain embodiments, the antibody will be purified (1) to greater than 95% by weight of protein as determined by the Lowry method, and alternatively, more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

“Cancer” or “tumor” is intended to include any malignant or neoplastic growth in a patient, including an initial tumor and any metastases. The cancer can be of the hematological or solid tumor type. Hematological tumors include tumors of hematological origin, including, e.g., myelomas (e.g., multiple myeloma), leukemias (e.g., Waldenstrom's syndrome, chronic lymphocytic leukemia, acute myelogenous leukemia, chronic myelogenous leukemia, granulocytic leukemia, monocytic leukemia, acute lymphocytic leukemia, other leukemias), lymphomas (e.g., B-cell lymphomas, such as diffuse large B-cell lymphoma, follicular lymphoma, mantle cell lymphoma, Hodgkin's lymphoma, non-Hodgkin's lymphoma, plasmocytoma, or reticulum cell sarcoma), and myeloproliferative neoplasms, such as myelodysplastic syndrome, thrombocythemia, polycythemia vera, or myelofibrosis. Solid tumors can originate in organs, and include cancers such as in skin, lung, brain, breast, prostate, ovary, colon, kidney, pancreas, liver, esophagus, stomach, intestine, bladder, uterus, cervix, testis, adrenal gland, etc. As used herein, cancer cells, including tumor cells, refer to cells that divide at an abnormal (increased) rate or whose control of growth or survival is different than for cells in the same tissue where the cancer cell arises or lives. Cancer cells include, but are not limited to, cells in carcinomas, sarcomas, myelomas, leukemias, lymphomas, and tumors of the nervous system including glioma, meningoma, medulloblastoma, schwannoma or epidymoma.

“Treatment” refers to therapeutic treatment. Those in need of treatment include those already with disease. Hence, the patient, e.g., human, to be treated herein may have been diagnosed as suffering from a disease, such as cancer or a nonmalignant hematological disease or suffering from the conditioning regimen. Alternatively, the patient may not have GvHD, but is a transplant patient, e.g., a patient undergoing conditioning for an allogeneic hematopoietic cell transplant, a candidate for or patient who is undergoing allogeneic hematopoietic cell transplant, e.g., allo-HSCT, or who underwent allogeneic hematopoietic cell transplant, e.g., allo-HSCT, recently, e.g., within the previous five months. Further, or alternatively, the patient may be planned to receive allogeneic T cells via donor leukocyte infusion (DLI) e.g., following allo-HSCT. Alternatively, a patient who received an allo-HSCT may suffer from acute GvHD or may have received corticosteroids for the treatment of GvHD. Treatment after allo-HSCT, e.g., after exhibiting symptoms of GvHD, may alleviate symptoms and may provide longer survival times.

A disease, e.g., cancer or GvHD is “inhibited” or “treated” if at least one symptom (as determined by responsiveness/non-responsiveness, or indicators known in the art and described herein) of the condition is alleviated, terminated, slowed, minimized, or prevented. The terms “patient” and “subject” are used interchangeably herein.

“Prevention” refers to a treatment that results in the absence or reduction in the severity of an adverse event. In a population of patients, treatment typically results in a certain percentage of adverse events, or a certain percentage of adverse events that are severe, but a treatment administered for prevention purposes instead results in a lower percentage of adverse events (i.e., a lower or reduced risk of adverse events) or a lower percentage of adverse events that are severe (i.e., a lower or reduced risk that the adverse event is severe).

In the context of allogeneic hematopoietic stem cell transplant patients, such as patients who undergo myeloablative or reduced-intensity conditioning and receive allogeneic hematopoietic stem cell transplants, the adverse event of graft-versus-host disease has at least a 25% risk, a 30% to 60% risk, a 35% to 55% risk, a 40% to 50% risk, or a 45% to 65% risk, and may result in 30% to 50% of the severe treatment related mortality that results from all adverse events. Prevention of the adverse GVHD, or prevention of high grade, e.g. grade III or IV or index C or D, GVHD may reduce the percent risk of the adverse event or may reduce the percent risk that GVHD leads to treatment related mortality of transplant patients. In some embodiments, the administration of an α4β7 antagonist, such as an anti-α4β7 antibody, prevents GVHD in a patient. In other embodiments, the administration of an α4β7 antagonist, such as an anti-α4β7 antibody, prevents the intestinal manifestation of GVHD in a patient. In some embodiments, the administration of an α4β7 antagonist, such as an anti-α4β7 antibody, prevents the intestinal manifestation of GVHD in a patient, but does not prevent one or more manifestations of GVHD in skin or liver. In some embodiments, the administration of an α4β7 antagonist, such as an anti-α4β7 antibody, reduces the use of immunosuppressive therapy in the patient. In some embodiments, the administration of an α4β7 antagonist, such as an anti-α4β7 antibody, to a patient undergoing allo-HSCT results in engraftment of the stem cells. In some embodiments, the administration of an α4β7 antagonist, such as an anti-α4β7 antibody, to a patient undergoing allo-HSCT results in engraftment of the stem cells and a graft-versus-tumor (GVT) effect.

The anti-α4β7 antibody is substantially pure and desirably substantially homogeneous (i.e. free from contaminating proteins etc.). “Substantially pure” antibody means a composition comprising at least about 90% antibody by weight, based on total weight of the protein in the composition, at least about 95% or 97% by weight. “Substantially homogeneous” antibody means a composition comprising protein wherein at least about 99% by weight of protein is specific antibody, e.g., anti-α4β7 antibody, based on total weight of the protein.

An anti-α4β7 antibody, vedolizumab, a humanized monoclonal antibody that has binding specificity for the α₄β₇ integrin, is already indicated for the treatment of patients with moderately to severely active ulcerative colitis (UC) and Crohn's disease (CD). Vedolizumab may also be used in the prevention of GvHD. Vedolizumab has a novel gut-selective mechanism of action. By binding to cell surface-expressed α₄β₇, vedolizumab is an α4β7 antagonist and blocks a subset of memory gut-homing T lymphocytes from interacting with mucosal addressin cell adhesion molecule-1 (MAdCAM-1) expressed on endothelial cells.

Several factors are associated with accelerated clearance of antibodies including the presence of anti-drug antibodies, sex, body size, concomitant immunosuppressant use, disease type, albumin concentration, and degree of systemic inflammation. Furthermore, a consistent relationship between efficacy and exposure, in distinction to drug dose, has been observed for many of these agents, such that higher trough drug concentrations are associated with greater efficacy. Differences in drug clearance may be an important explanation for this observation. For example, cancer patients undergo immunosuppressive treatment of the tumor and treatment for infection. Therefore, an understanding of the determinants of clearance for therapeutic antibodies in transplant patients may result in optimization of drug regimens.

In previous studies, single-dose pharmacokinetics, pharmacodynamics (α₄β₇ receptor saturation), safety, and tolerability of vedolizumab were investigated over a dose range of 0.2 to 10 mg/kg in healthy volunteers (intravenous [IV] infusion) (unpublished data). After reaching peak concentrations, vedolizumab serum concentrations fell in a generally biexponential fashion until concentrations reached approximately 1 to 10 ng/mL. Thereafter, concentrations appeared to fall in a nonlinear fashion. The multiple-dose pharmacokinetics and pharmacodynamics of vedolizumab have been investigated following IV infusions of 0.5 and 2 mg/kg in patients with CD and infusion of 2, 6, and 10 mg/kg in patients with UC. Vedolizumab pharmacokinetics was generally linear following an IV infusion over the dose range of 2 to 10 mg/kg in patients with UC. After multiple-dose administration, rapid and near complete α₄β₇ receptor saturation was achieved following the initial dose of vedolizumab.

The efficacy and safety of vedolizumab induction and maintenance therapy were demonstrated in patients with CD in the GEMINI 2 (ClinicalTrials.gov number, NCT00783692) and GEMINI 3 (ClinicalTrials.gov number, NCT01224171) trials. The exposure-response (efficacy) relationships of vedolizumab in patients with CD for induction and maintenance therapy have been presented elsewhere.

The invention relates to a method of treating disease in a patient by preventing GvHD, or a GvHD-related adverse event, in a allogeneic hematopoietic cell transplant patient, e.g., human patient, e.g., undergoing allo-HSCT. The human patient may be an adult (e.g., 18 years or older), an adolescent, or a child. A pharmaceutical composition comprising an anti-α4β7 antibody can be used as described herein for treating a transplant patient, a cancer patient, a nonmalignant hematological disease patient or preventing GvHD in a subject suffering therefrom.

The severity of acute GvHD is measured according to the modified Glucksberg criteria (Table 2) and Blood and Marrow Transplant Clinical Trials Network (BMT CTN)-modified International Bone Marrow Transplant Registry Database (IBMTR) index Table 3). The clinical stages and grades of GvHD are divided as shown in Table 1.

TABLE 1
Acute Graft-versus-Host Disease Clinical Stage

| | | Liver | | |
|---|---|---|---|---|
| | | Bilirubin: SI units | | Intestinal tract |
| Stage | Skin | (standard units) | | Diarrhea/day |
| 1 | Maculopapular rash <25% of | 34-50 | μmol/L | >500 mL |
| | body surface (a) | (2-3 | mg/dL) | diarrhea/day |
| 2 | Maculopapular rash 25%-50% | 51-102 | μmol/L | >1000 mL |
| | of body surface | (3.1-6 | mg/dL) | diarrhea/day |
| 3 | Rash >50% of body surface | 103-225 | μmol/L | >1500 mL |
| | | (6.1-15 | mg/dL) | diarrhea/day |
| 4 | Generalized erythroderma with | >255 | μmol/L | Severe abdominal |
| | bullous formation | (>15 | mg/dL) | pain, with or without |
| | | | | ileus |

TABLE 2
Acute Graft-versus-Host Disease Grade (modified Glucksberg)

| Grade | Skin | Liver | Intestinal tract |
|---|---|---|---|
| I | Stage 1-2 | None | None |
| II | Stage 3 or → | Stage 1 or → | Stage 1 |
| III | — | Stage 2-3 or → | Stage 2-4 |
| IV | Stage 4 or → | Stage 4 | — |

TABLE 3
Criteria for International Bone Marrow
Transplant Registry Database (IBMTR)
Severity Index for Acute Graft-versus-Host Disease

| | Skin | | | Liver | | | Intestinal tract | |
|---|---|---|---|---|---|---|---|---|
| | | Extent | | | Total | | | Volume of |
| | Stage | of | | Stage | Bilirubin | | Stage | Diarrhea |
| Index | (max) | Rash | | (max) | (μmol/L) | | (max) | (mL/day) |
| A | 1 | <25% | | 0 | <34 | | 0 | <500 |
| B | 2 | 25-50% | or | 1-2 | 34-102 | or | 1-2 | 550-1500 |
| C | 3 | >50% | or | 3 | 103-255 | or | 3 | <1500 |
| D | 4 | Bullae | or | 4 | >255 | or | 4 | Severe |
| | | | | | | | | pain and |
| | | | | | | | | ileus |

The allogeneic hematopoietic cells, e.g., allo-HSC, may engraft with no GvHD, only skin GvHD, only liver GvHD, only skin and liver GvHD, no intestinal GvHD and only skin or liver GvHD, no grade IV GvHD, no grade III or IV GvHD, only stage 1 or stage 2 intestinal GvHD and only stage 2-3 skin and/or liver GvHD, only Grade I to II GvHD, or no or only skin GvHD, only index A GvHD, only index A or B GvHD, no index C or D GvHD, or any of the foregoing together with GVT, after administration of the α4β7 antagonist, e.g., an anti-α4β7 antibody.

Preventing the development of acute GvHD may be the result of decreasing or blocking trafficking of alloreactive T-cells to GALT, mesenteric lymph nodes and/or GI mucosa. Prevention of GvHD, e.g., acute GvHD, may be considered successful if at about 50 days, about 75 days, about 90 days, about 100 days, about 110 days, about 120 days, about 150 days, or about 180 days, after allogeneic hematopoietic cell transplant, e.g., allo-HSCT, the patient shows no signs of acute GvHD. In some embodiments, the patient undergoing allogeneic hematopoietic cell transplant, e.g., allo-HSCT is treated with a regimen that comprises no further administration of immunosuppressive therapy, e.g., no administration of immunosuppressive therapy after the conditioning treatment or after the initial transplant period, e.g., immediately before and/or immediately after, e.g., 0 to 1 weeks, 0 to 2 weeks, 0 to 3 weeks or 0 to 4 weeks, after the allogeneic hematopoietic cell transplant.

Remission is defined by conventional World Health Organization (WHO) criteria: <5% blast cells, count recovery, and no evidence of extramedullary disease. Remission of acute and/or chronic GvHD may last for about 4, about 5, about 6, about 9, or about 12 months after allo-HSCT.

GvHD relapse or progression-free survival (GRFS) is defined as Grade 3-4 acute GvHD, chronic GvHD requiring systemic immunosuppression, disease relapse or progression, or death due to any cause.

Engraftment is a process whereby the transplanted hematopoietic cells populate in the patient or adjust to the patient tissue environment, e.g., proliferate, differentiate, and begin performing the function characteristic of the hematologic cell from which it is derived or is programmed to become with the maturation signals. Engraftment of allo-HSCT is measured by quantifying blood components, such as neutrophils and platelets. The timing of engraftment depends on the source of the hematopoietic stem cells, e.g., longer for cord blood stem cells than for peripheral blood stem cells. Neutrophil engraftment (recovery of absolute neutrophil count [ANC]) is defined by an ANC>500/mm³ for 3 consecutive days or >2000/mm³ for 1 day. The first day of the 3-day period is considered the day of neutrophil engraftment.

The mean expression of α4β7 on peripheral blood lymphocytes may be measured by the MadCAM-1-Fc binding inhibition assay before and after dosing with an anti-α4β7 antibody (e.g., vedolizumab) in the allogeneic hematopoietic cell transplant patient, e.g., myeloablative allo-HSCT population.

Changes in blood or serum biomarkers, including, but not limited to, interleukin-6 (IL-6), interleukin-17 (IL-17), and suppressor of tumorigenicity 2 (ST2) and/or cellular biomarkers, including, but not limited to CD8+, CD38+, CD8+ bright effector memory T cells, and CD4+ memory T cells, may be predictive of the onset or severity of acute GvHD. Detection of an increase one or more of such markers after allo-HSCT may indicate the onset of acute GVHD. Detection of the biomarkers may be accomplished from immunodetection of the biomarker, e.g., by antibody binding to cells, e.g., blood cells, expressing the biomarker and measurement of the amount of antibody binding, e.g., by flow cytometry or by antibody binding to soluble biomarkers in serum and measurement of the amount of antibody binding, e.g., by ELISA. Comparison of the amount of the biomarker with a control or a sample obtained early in the transplant process or prior to transplant, or to a predetermined standard, e.g., the amount of the biomarker in a population of non-transplant subjects, may provide an indication of whether the amount of the biomarker is changed, e.g., increased. In some embodiments, administration of an α4β7 antagonist, such as an anti-α4β7 antibody, to a patient undergoing allogeneic hematopoietic cell transplant, e.g., allo-HSCT, prevents a change or an increase in one or more of these biomarkers.

Patients may be tested to see if they are positive for antibodies directed against the α4β7 antagonist, such as anti-α4β7 antibody, for example, positive for anti-vedolizumab antibody at various time points, for example, at baseline, day 20, and day 100 after allo-HSCT.

Patients may be tested for development of GvHD requiring systemic immunosuppression.

An α4β7 antagonist, such as an anti-α4β7 antibody, is administered in an effective amount which inhibits binding of α4β7 integrin to a ligand thereof. For therapy, an effective amount will be sufficient to achieve the desired prophylactic effect (e.g., decreasing or eliminating trafficking of alloreactive T-cells to GALT, mesenteric lymph nodes and or GI mucosa and reducing the incidence or severity of GvHD). An effective amount of an anti-α4β7 antibody, e.g., an effective titer sufficient to maintain saturation, e.g., neutralization, of α4β7 integrin, can result in sustained α4β7 blockade at the time of hematopoietic stem cell infusion. An α4β7 antagonist, such as an anti-α4β7 antibody may be administered in a unit dose or multiple doses. The dosage can be determined by methods known in the art and can be dependent, for example, upon the individual's age, sensitivity, tolerance and overall well-being. Examples of modes of administration include topical routes such as nasal or inhalational or transdermal administration, enteral routes, such as through a feeding tube or suppository, and parenteral routes, such as intravenous, intramuscular, subcutaneous, intra-arterial, intraperitoneal, or intravitreal administration. Suitable dosages for antibodies can be from about 0.1 mg/kg body weight to about 10.0 mg/kg body weight per treatment, for example about 2 mg/kg to about 7 mg/kg, about 3 mg/kg to about 6 mg/kg, or about 3.5 to about 5 mg/kg. In particular embodiments, the dose administered is about 0.3 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In some embodiments, vedolizumab is administered at a dose of 50 mg, 75 mg, 100 mg, 300 mg, 450 mg, 500 mg or 600 mg. In some embodiments, vedolizumab is administered at a dose of 108 mg, 90 to 120 mg, 216 mg, 160 mg, 165 mg, 155 to 180 mg, 170 mg or 180 mg. In some embodiments, vedolizumab is administered at a dose of 180 to 250 mg, 300 to 350 mg, or 300 to 500 mg.

In the case of an α4β7 antagonist, such as an anti-α4β7 antibody which is stored as a lyophilized solid, the antibody is reconstituted in a solution such as water for injection prior to administration. If prepared for infusion, the final dosage form, e.g., after dilution of the reconstituted antibody (e.g., in a saline, Ringer's or 5% dextrose infusion system) of the anti-α4β7 antibody can be about 0.5 mg/ml to about 5 mg/ml for administration. The final dosage form may be at a concentration of between about 0.3 mg/ml to about 3.0 mg/ml, about 1.0 mg/ml to about 1.4 mg/ml, about 1.0 mg/ml to about 1.3 mg/ml, about 1.0 mg/ml to about 1.2 mg/ml, about 1.0 to about 1.1 mg/ml, about 1.1 mg/ml to about 1.4 mg/ml, about 1.1 mg/ml to about 1.3 mg/ml, about 1.1 mg/ml to about 1.2 mg/ml, about 1.2 mg/ml to about 1.4 mg/ml, about 1.2 mg/ml to about 1.3 mg/ml, or about 1.3 mg/ml to about 1.4 mg/ml. The final dosage form may be at a concentration of about 0.6 mg/ml, 0.8 mg/ml, 1.0 mg/ml, 1.1 mg/ml, about 1.2 mg/ml, about 1.3 mg/ml, about 1.4 mg/ml, about 1.5 mg/ml, about 1.6 mg/ml, about 1.8 mg/ml or about 2.0 mg/ml. In one embodiment, the total dose is 75 mg. In one embodiment, the total dose is 150 mg, 225 mg, 375 mg or 525 mg. In another embodiment, the total dose is 300 mg. In one embodiment, the total dose is 450 mg. In one embodiment, the total dose is 600 mg. An anti-α4β7 antibody dose may be diluted into 250 ml saline, Ringer's or 5% dextrose solution for administration.

The dose can be administered to the patient over about 20 minutes, about 25 minutes, about 30 minutes, about 35 minutes, or about 40 minutes.

The dosing regimen can be optimized to result in the prevention of GvHD or the reduction of the risk of severe Grade or index level, e.g., Grade III or IV, index C or index D of GvHD suffered by the patient. In some embodiments, the dosing regimen does not alter the ratio of CD4 to CD8 in cerebrospinal fluid of patients receiving treatment. For example, the anti-α4β7 antagonist does not impair immune surveillance of the nervous system, e.g., the brain or spinal cord.

In one embodiment, the dosing regimen comprises an initial dose the day before an allogeneic stem cell transplantation (allo-HSCT), a subsequent dose approximately two weeks after the initial dose, and a second subsequent dose approximately six weeks after the initial dose. In an embodiment, the initial dose of the anti-α4β7 antibody is at least 12 hours before the allogeneic stem cell infusion. Although this anti-α4β7 antibody dosing regimen is useful for the induction dose and schedule of vedolizumab approved for the treatment of Crohn's Disease or ulcerative colitis, subjects undergoing an allogeneic hematopoietic cell transplant, such as being treated with a conditioning regimen followed by the transplant, e.g., allo-HSCT, are expected to have markedly changing T-cell populations with variable α4β7 integrin expression during the post-transplant period. Furthermore, if the patient contracts infections or GVHD or has other adverse effects from the transplant procedure, clearance of the anti-α4β7 antibody may be affected. For example, if kidney damage results from the agents used for conditioning, treatment with dialysis could increase the clearance of antibodies from the bloodstream. Alternatively, after myeloablative therapy, there may be other physiological conditions that may result in unexpectedly high clearance of the anti-α4β7 antibody during initial therapy.

In some embodiments, an anti-α4β7 antibody is administered prior to allogeneic hematopoietic cell transplant, e.g., allo-HSCT. In some embodiments, an α4β7 antagonist, such as an anti-α4β7 antibody, is administered to a patient prior to and after allogeneic hematopoietic cell transplant, e.g., allo-HSCT. In some embodiments, an α4β7 antagonist, such as an anti-α4β7 antibody, is administered to a patient after allogeneic hematopoietic cell transplant, e.g., allo-HSCT, e.g., within 1 day after, 1 to 2 days after, 1 to 3 days after, 2 to 3 days after or 2 to 4 days after, 2 days after, 3 days after, 4 days after, 5 days after, 6 days after or 7 days after allogeneic hematopoietic cell transplant, e.g., allo-HSCT. In some embodiments, an anti-α4β7 antibody is administered to a patient 1 to 100 days, 5 to 80 days, 5 to 30 days, 10 to 28 days, 10 to 50 days, 14 to 30 days, 15 to 32 days, 18 to 25 days, 15 to 35 days or greater than 100 days after allo-HSCT. For example, an anti-α4β7 antibody, e.g., vedolizumab, may be administered by intravenous infusion as an initial dose the day before allogeneic hematopoietic cell transplant, e.g., allo-HSCT, and then again at two, and six weeks after the initial dose.

In a particular aspect, the present invention provides a method for preventing GvHD in an allogeneic hematopoietic cell transplant, e.g., allogeneic hematopoietic stem cell transplant patient, using vedolizumab. The method comprises the steps of administering an initial 300 mg dose of an anti-α4β7 antibody (vedolizumab) to a hematologic cancer patient, such as a person suffering from leukemia, performing an allo-HSCT one day after the initial dose of vedolizumab, administering a subsequent 300 mg dose of vedolizumab two weeks after the initial dose, and a second subsequent 300 mg dose of vedolizumab six weeks after the initial dose. Alternatively, in some embodiments, the dose of the anti-α4β7 antibody (vedolizumab) is lower (e.g., 75 mg or 150 mg) or higher (e.g., 450 mg or 600 mg) than 300 mg.

The invention provides an anti-α4β7 antibody for use in preventing GVHD in a patient having an allogeneic hematopoietic cell transplant, e.g., allo-HSCT, the use comprising administering an initial dose of the anti-α4β7 antibody the day before the allo-HSCT, two weeks after the initial dose, and six weeks after the initial dose. The use in preventing may further comprise administration of tacrolimus and/or methotrexate. In some embodiments, the anti-α4β7 antibody is vedolizumab.

This disclosure also relates to methods for treating GvHD by administering an effective amount of an antagonist of human α4β7 integrin, such as an anti-α4β7 antibody (e.g., vedolizumab), to a subject in need thereof. The method is particularly useful for treating acute GvHD, and steroid refractory acute GvHD. An example of steroid refractory acute GvHD, is steroid refractory acute GvHD with intestinal disease involvement, for example, with a severity index of B, C or D (using the BMT CTN-modified IBMTR index), an ECOC performance status of 0 to 3, and/or a creatinine clearance of ≥60 mL/minute/1.73 m² (based on the Cockcroft-Gault estimate). A steroid refractory patient may have worsening or no improvement in 5 to 7 days of treatment with a corticosteroid, e.g., cortisone, hydrocortisone, prednisone or methylprednisolone, or have received an increase in dose of corticosteroid. The method or treatment is particularly useful for treating GvHD in a patient who has received allo-HSCT, including a patient that has evidence of myeloid engraftment.

For the treatment of GvHD (including steroid refractory acute GvHD), an antibody that has binding specificity for human α4β7 integrin (e.g., vedolizumab) may be administered in one or more doses of about 300 mg, 350 mg, 400 mg, 450 mg, 500 mg, 550 mg or 600 mg of antibody, e.g., doses of 300 mg or 600 mg. Each does that is administered to the patient may contain the same amount of antibody, for example multiple doses of 300 mg of antibody (vedolizumab) or multiple doses of 600 mg of antibody (vedolizumab) can be administered.

The antibody that has binding specificity for human α4β7 integrin can be administered according to an administration regimen. One regimen includes a) administering a first dose of antibody; b) administering a second dose of antibody about two weeks after the first dose; and c) administering a third dose of antibody about four weeks after the second dose. Optionally, further doses of antibody can be administered, with the proviso that each further dose is administered about four weeks after the immediate prior dose. In some embodiments, each dose that is administered according to the regimen contains about 300 mg of antibody (e.g., vedolizumab), or each dose each does contains about 600 mg of antibody (e.g., vedolizumab).

The antibody that has binding specificity for human α4β7 integrin is administered to the patient in need thereof intravenously, for example by intravenous infusion. When administered by intravenous infusion, the infusion can be over a period of about 30 minutes to about 60 minutes.

A pharmaceutical composition comprising an anti-α4β7 antibody can be used as described herein for treating a transplant patient, a cancer patient, a nonmalignant hematological disease patient or preventing GvHD in a subject suffering therefrom. A pharmaceutical composition comprising an anti-α4β7 antibody can also be used as described herein to treat GvHD (including steroid refractory acute GvHD). An α4β7 antagonist, such as an anti-α4β7 antibody, is administered in an effective amount which inhibits binding of α4β7 integrin to a ligand thereof.

The methods described herein comprise administering an effective amount of an anti-α4β7 antibody to a patient. If the anti-α4β7 antibody is in a formulation which is in a solid, e.g., dry state, the process of administration can comprise a step of converting the formulation to a liquid state. In one aspect, a dry formulation can be reconstituted, e.g., by a liquid as described above, for use in injection, e.g. intravenous, intramuscular or subcutaneous injection. In another aspect, a solid or dry formulation can be administered topically, e.g., in a patch, cream, aerosol or suppository.

The α4β7 antagonist, which is an anti-α4β7 antibody, can bind to an epitope on the α4 chain (e.g., humanized MAb 21.6 (Bendig et al., U.S. Pat. No. 5,840,299), on the β7 chain (e.g., FIB504 or a humanized derivative (e.g., Fong et al., U.S. Pat. No. 7,528,236)), or to a combinatorial epitope formed by the association of the α4 chain with the β7 chain. AMG-181 or other antibodies described in US 2010/0254975 are anti-α4β7 antibodies. In one aspect, the antibody binds a combinatorial epitope on the α4β7 complex, but does not bind an epitope on the α4 chain or the β7 chain unless the chains are in association with each other. The association of α4 integrin with β7 integrin can create a combinatorial epitope for example, by bringing into proximity residues present on both chains which together comprise the epitope or by conformationally exposing on one chain, e.g., the α4 integrin chain or the β7 integrin chain, an epitopic binding site that is inaccessible to antibody binding in the absence of the proper integrin partner or in the absence of integrin activation. In another aspect, the anti-α4β7 antibody binds both the α4 integrin chain and the β7 integrin chain, and thus, is specific for the α4β7 integrin complex. The anti-α4β7 antibody can bind α4β7 but not bind α4β1, and/or not bind α_{E}β7, for example. In another aspect, the anti-α4β7 antibody binds to the same or substantially the same epitope as the Act-1 antibody (Lazarovits, A. I. et al., J. Immunol., 133(4): 1857-1862 (1984), Schweighoffer et al., J. Immunol., 151(2): 717-729, 1993; Bednarczyk et al., J. Biol. Chem., 269(11): 8348-8354, 1994). Murine ACT-1 Hybridoma cell line, which produces the murine Act-1 monoclonal antibody, was deposited under the provisions of the Budapest Treaty on Aug. 22, 2001, on behalf Millennium Pharmaceuticals, Inc., 40 Landsdowne Street, Cambridge, Mass. 02139, U.S.A., at the American Type Culture Collection, 10801 University Boulevard, Manassas, Va. 20110-2209, U.S.A., under Accession No. PTA-3663. In another aspect, the anti-α4β7 antibody is a human antibody or an α4β7 binding protein using the CDRs provided in U.S. Patent Application Publication No. 2010/0254975.

In one aspect, the α4β7 antagonist is an anti-MAdCAM antibody (see e.g., U.S. Pat. No. 8,277,808, PF-00547659 or antibodies described in WO2005/067620), or an engineered form of a ligand, such as a MAdCAM-Fc chimera such as described in U.S. Pat. No. 7,803,904.

In one aspect, the anti-α4β7 antibody inhibits binding of α4β7 to one or more of its ligands (e.g. the mucosal addressin, e.g., MAdCAM (e.g., MAdCAM-1), fibronectin, and/or vascular addressin (VCAM)). Primate MAdCAMs are described in the PCT publication WO 96/24673, the entire teachings of which are incorporated herein by this reference. In another aspect, the anti-α4β7 antibody inhibits binding of α4β7 to MAdCAM (e.g., MAdCAM-1) and/or fibronectin without inhibiting the binding of VCAM.

In one aspect, the anti-α4β7 antibodies for use in the treatments are humanized versions of the mouse Act-1 antibody. Suitable methods for preparing humanized antibodies are well-known in the art. Generally, the humanized anti-α4β7 antibody will contain a heavy chain that contains the 3 heavy chain complementarity determining regions (CDRs, CDR1, SEQ ID NO:4, CDR2, SEQ ID NO:5 and CDR3, SEQ ID NO:6) of the mouse Act-1 antibody and suitable human heavy chain framework regions; and also contain a light chain that contains the 3 light chain CDRs (CDR1, SEQ ID NO:7, CDR2, SEQ ID NO:8 and CDR3, SEQ ID NO:9) of the mouse Act-1 antibody and suitable human light chain framework regions. The humanized Act-1 antibody can contain any suitable human framework regions, including consensus framework regions, with or without amino acid substitutions. For example, one or more of the framework amino acids can be replaced with another amino acid, such as the amino acid at the corresponding position in the mouse Act-1 antibody. The human constant region or portion thereof, if present, can be derived from the κ or λ light chains, and/or the γ (e.g., γ1, γ2, γ3, γ4), μ, α (e.g., α1, α2), δ or ε heavy chains of human antibodies, including allelic variants. A particular constant region (e.g., IgG1), variant or portions thereof can be selected in order to tailor effector function. For example, a mutated constant region (variant) can be incorporated into a fusion protein to minimize binding to Fc receptors and/or ability to fix complement (see e.g., Winter et al., GB 2,209,757 B; Morrison et al., WO 89/07142; Morgan et al., WO 94/29351, Dec. 22, 1994). Humanized versions of Act-1 antibody were described in PCT publications nos. WO98/06248 and WO07/61679, the entire teachings of each of which are incorporated herein by this reference. Treatment methods using anti-α4β7 integrin antibodies are described in publication nos. U.S. 2005/0095238, U.S. 2005/0095238, WO2012151248 and WO 2012/151247.

In one aspect, the anti-α4β7 antibody is vedolizumab. Vedolizumab IV (also called MLN0002, ENTYVIO™ or KYNTELES™) is a humanized antibody (IgG1 mAb) directed against the human lymphocyte integrin α4β7. The α4β7 integrin mediates lymphocyte trafficking to GI mucosa, gut-associated lymphoid tissue (GALT) and mesenteric lymph nodes through adhesive interaction with mucosal addressin cell adhesion molecule-1 (MAdCAM-1), which is expressed on the endothelium of mesenteric lymph nodes and GI mucosa. Vedolizumab binds the α4β7 integrin, antagonizes its adherence to MAdCAM-1 and as such, impairs the migration of naïve T cells to the GALT and mesenteric lymph nodes and gut homing leukocytes into GI mucosa.

In another aspect, the humanized anti-α4β7 antibody for use in the treatment comprises a heavy chain variable region comprising amino acids 20 to 140 of SEQ ID NO:1, and a light chain variable region comprising amino acids 20 to 131 of SEQ ID NO:2 or amino acids 1 to 112 of SEQ ID NO:3. If desired, a suitable human constant region(s) can be present. For example, the humanized anti-α4β7 antibody can comprise a heavy chain that comprises amino acids 20 to 470 of SEQ ID NO:1 and a light chain comprising amino acids 1 to 219 of SEQ ID NO:3. In another example, the humanized anti-α4β7 antibody can comprise a heavy chain that comprises amino acids 20 to 470 of SEQ ID NO:1 and a light chain comprising amino acids 20 to 238 of SEQ ID NO:2. Vedolizumab is cataloged under Chemical Abstract Service (CAS, American Chemical Society) Registry number 943609-66-3).

Substitutions to the humanized anti-α4β7 antibody sequence can be, for example, mutations to the heavy and light chain framework regions, such as a mutation of isoleucine to valine on residue 2 of SEQ ID NO:10; a mutation of methionine to valine on residue 4 of SEQ ID NO:10; a mutation of alanine to glycine on residue 24 of SEQ ID NO:11; a mutation of arginine to lysine at residue 38 of SEQ ID NO:11; a mutation of alanine to arginine at residue 40 of SEQ ID NO:11; a mutation of methionine to isoleucine on residue 48 of SEQ ID NO:11; a mutation of isoleucine to leucine on residue 69 of SEQ ID NO:11; a mutation of arginine to valine on residue 71 of SEQ ID NO:11; a mutation of threonine to isoleucine on residue 73 of SEQ ID NO:11; or any combination thereof; and replacement of the heavy chain CDRs with the CDRs (CDR1, SEQ ID NO:4, CDR2, SEQ ID NO:5 and CDR3, SEQ ID NO:6) of the mouse Act-1 antibody; and replacement of the light chain CDRs with the light chain CDRs (CDR1, SEQ ID NO:7, CDR2, SEQ ID NO:8 and CDR3, SEQ ID NO:9) of the mouse Act-1 antibody.

The α4β7 antagonist, such as anti-α4β7 antibody may be administered to an individual (e.g., a human) alone or in conjunction with another agent. The α4β7 antagonist, such as an anti-α4β7 antibody can be administered before, along with or subsequent to administration of the additional agent. In one embodiment, more than one α4β7 antagonist which inhibits the binding of α4β7 integrin to its ligands is administered. In such an embodiment, an agent, e.g., a monoclonal antibody, such as an anti-MAdCAM (e.g., anti-MAdCAM-1) or an anti-VCAM-1 monoclonal antibody can be administered. In another embodiment, the additional agent inhibits the binding of leukocytes to an endothelial ligand in a pathway different from the α4β7 pathway. Such an agent can inhibit the binding, e.g. of chemokine (C—C motif) receptor 9 (CCR9)-expressing lymphocytes to thymus expressed chemokine (TECK or CCL25) or an agent which prevents the binding of LFA-1 to intercellular adhesion molecule (ICAM). For example, an anti-TECK or anti-CCR9 antibody or a small molecule CCR9 inhibitor, such as inhibitors disclosed in PCT publication WO03/099773 or WO04/046092, or anti-ICAM-1 antibody or an oligonucleotide which prevents expression of ICAM, is administered in addition to a formulation of the present invention. In yet another embodiment, one or more additional active ingredients (e.g., methotrexate or a calcineurin inhibitor, e.g., tacrolimus or cyclosporin) commonly administered for GvHD prophylaxis therapy, may be administered in conjunction with an α4β7 antagonist, such as an anti-α4β7 antibody in a method of the present invention. In an embodiment, the dose of the co-administered medication can be decreased over time during the period of treatment by the α4β7 antagonist, such as an anti-α4β7 antibody.

In some embodiments, the co-administered medication is a calcineurin inhibitor, such as tacrolimus. In some embodiments, the calcineurin inhibitor treatment is started before allogeneic hematopoietic cell transplant, e.g., allo-HSCT and continued until at least day 100. In one embodiment, tacrolimus treatment may start during conditioning for the allogeneic hematopoietic cell transplant, e.g., allo-HSCT. The tacrolimus treatment may achieve a trough concentration of about 1 ng/dL, about 2 ng/dL, about 3 ng/dL, about 4 ng/dL, about 5 ng/dL, about 6 ng/dL, about 7 ng/dL, about 8 ng/dL, about 9 ng/dL, about 10 ng/dL, or about 5-10 ng/dL. Tacrolimus treatment may be kept at therapeutic levels for about 2 weeks, about 6 weeks, about 2 months, about 3 months, about 100 days after allogeneic hematopoietic cell transplant, e.g., allo-HSCT if no signs of GvHD are observed. Tacrolimus treatment may be discontinued by about 5 months, about 6 months, about 7 months after allogeneic hematopoietic cell transplant, e.g., allo-HSCT.

In some embodiments, the co-administered medication is methotrexate. In an embodiment, methotrexate is administered to the patient at about 2, 4, 6, 8, 10, or 12 mg/m² IV after allogeneic hematopoietic cell transplant, e.g., allo-HSCT (e.g., on days 1, 3, 6, and 11). The amount of methotrexate administered to the patient may be modified, or held, based on toxicity.

The invention will be more fully understood by reference to the following examples. They should not, however, be construed as limiting the scope of the invention. All literature and patent citations are incorporated herein by reference.

### EXEMPLIFICATION

### Example 1

A phase 1b, open-label, dose-finding study is designed to evaluate the safety, tolerability, and clinical activity of adding vedolizumab to standard graft-versus-host disease (GvHD) prophylaxis (tacrolimus plus short-term methotrexate) in adult patients undergoing allogeneic hematopoietic stem cell transplantation (allo-HSCT). Vedolizumab dose finding is cohort based and follows a rule-based dose-finding study design with pharmacokinetic (PK) guidance. After a tolerated dose with acceptable PK is identified, the cohort at that dose level may be expanded to further assess the tolerability and effectiveness of vedolizumab.

Eligibility is determined during the Screening period, which may last for up to 28 days before Day −1 (designation of the day of the first IV infusion of vedolizumab). Patients who meet all eligibility criteria and provide written informed consent are enrolled in this study. Study drug is administered initially on Day −1 before allo-HSCT and then on Days +13 and +42 after allo-HSCT. Patients who are undergoing unrelated-donor myeloablative transplant for the treatment of hematologic malignancies and who are less than or equal to 60 years of age are eligible for enrollment. After a recommended phase 2 dose is identified, the cohort at that dose level can be expanded to include additional patients receiving myeloablative conditioning or reduced-intensity conditioning “RIC” (less than or equal to 75 years of age) who are undergoing either related or unrelated allogeneic HSCT for the treatment of hematologic malignancies or myeloproliferative neoplasms.

Patients are excluded from the study if they have received prior allogeneic transplants or if they planned to undergo umbilical cord blood transplant, receive ex vivo T-cell-depleted hematopoietic stem cells (HSCs), receive any in vivo T-cell depleting antibodies, or RIC (in the dose-finding portion only). Patients with active cerebral/meningeal disease, active cytomegalovirus (CMV) colitis, or signs and symptoms of progressive multifocal leukoencephalopathy (PML) or any history of PML are also excluded. In addition, patients with nonmalignant hematological disorders (e.g., aplastic anemia, sickle cell anemia, thalassemias, Fanconi anemia) are excluded in both portions of the study.

For PK endpoints, an evaluable patient is one who receives vedolizumab and has at least 1 PK sample collected.

Patients who remain in remission are followed for safety and development of acute and chronic GvHD for 1 year after allo-HSCT or until the patient's death or withdrawal of consent or termination of the study by the sponsor. All patients are followed for overall survival (OS) until death, withdrawal of consent, termination of the study by the sponsor, or for a maximum of 1 year after the last patient is enrolled in the study. Patients attend a Day +100 visit (±7 days) at which time they will enter posttreatment follow-up.

Dose escalation starts with a low-dose cohort receiving vedolizumab at 75 mg IV on Day −1 and on Days +13 and +42 after allo-HSCT. HSC infusion occurs on Day 0 (no sooner than 12 hours after completion of IV infusion of vedolizumab on Day −1). The first patient in each dosing cohort is monitored for dose-limiting toxicities (DLTs) from the start of the first IV infusion of vedolizumab on Day −1 to Day +28 after allo-HSCT (the DLT observation period) including assessment for neutrophil recovery by Day +28. If the first patient in the first cohort tolerates vedolizumab IV at 75 mg and engraftment occurs, then 2 more patients will be enrolled in the first cohort. If none of the first 3 patients experience DLTs, the next cohort receives vedolizumab 300 mg IV on Day −1 and on Days +13 and +42 after allo-HSCT. If the first patient in this cohort tolerates vedolizumab IV at 300 mg and engraftment occurs, then 2 more patients are enrolled in the second cohort. If the first 3 patients at 300 mg tolerate the treatment without experiencing DLTs, then the decision on whether to increase the vedolizumab IV dose in the next cohort is guided by the PK results. If 1 of the first 3 patients in the cohort experiences a DLT, then 3 additional patients are enrolled at the same dose level and monitored for DLTs from Day −1 until Day +28. If none of the additional patients experiences a DLT, then the decision on whether to increase the vedolizumab IV dose in the next cohort is guided by the PK results. If 2 or more patients in a cohort of either 3 or 6 patients experience a DLT, then the dose of vedolizumab IV for the next cohort of 3 patients is reduced. These patients will be monitored for DLTs in the same manner that patients in the previous cohort were monitored.

After a tolerated dose level with acceptable PK is identified in patients who are undergoing unrelated-donor myeloablative transplant for the treatment of hematologic malignancies, the cohort at that dose level may be expanded to include approximately 18 additional patients undergoing myeloablative conditioning or reduced-intensity conditioning (RIC) and are receiving either related or unrelated allo-HSCT for the treatment of hematologic malignancies or myeloproliferative neoplasms. This group of patients allows for the further assessment of the tolerability and clinical activity of vedolizumab IV.

Vital signs, physical and neurological examinations, adverse event (AE) assessments, and laboratory values (chemistry, hematology, and urinalysis) are obtained to evaluate the safety and tolerability of vedolizumab IV. To exclude patients with progressive multifocal leukoencephalopathy (PML), a Risk Assessment and Minimization for PML (RAMP) questionnaire is administered at Screening and before vedolizumab IV administration on Days −1 before allo-HSCT, and on Days +13 and +42 after allo-HSCT. Serial blood samples for the evaluation of PK of vedolizumab are obtained at prespecified time points. PK of vedolizumab is analyzed for each of the first 3 patients at each dose level. It is expected that the concentration-time profile of vedolizumab will be influenced by the level of α₄β₇ target saturation. If α₄β₇ is saturated, then vedolizumab clearance would be linear; if α₄β₇ is not saturated, then clearance would be nonlinear indicating rapid elimination. If the clearance of vedolizumab is nonlinear at the 300 mg dose, then subsequent dosing for all patients is increased in approximately 150 mg increments (up to a maximum of 600 mg) until linear PK clearance is achieved.

Serial blood samples for determination of the serum concentration of vedolizumab and anti-vedolizumab antibodies and serum biomarkers (including, but not limited to, interleukin-6 [IL-6], interleukin-17 [IL-17], and suppressor of tumorigenicity 2 [ST2]) are obtained at pre-specified time points. In addition, blood samples will be collected to perform flow cytometry for cell immunophenotyping to measure cell populations as determined by levels of various cellular biomarkers (such as CD8+, CD38+, CD8+ effector memory T cells, and CD4+ memory T cells), and to perform MadCAM-1-FC binding inhibition assays at pre-specified time points.

Toxicity is evaluated according to National Cancer Institute Common Terminology Criteria for Adverse Events (NCI CTCAE), Version 4.03, effective date 14 Jun. 2010.

### Example 2 Treatment of Graft Versus Host Disease

An open-label phase 2a study is conducted to assess the tolerability and effectiveness of intravenously administered vedolizumab for the treatment of graft versus host disease in patients who have undergone allogeneic hematopoietic stem cell transplantation (allo-HSCT). The study will also be used to identify a recommended dose and regimen of intravenously administered vedolizumab for this indication. The study will enroll approximately 38 participants, who will be randomized at a ratio of 1:1 to 2 treatment arms to receive either 300 mg or 600 mg vedolizumab IV on Days 1, 15, 43, 71, and 99.

### A. Description of Investigational Agent

The vedolizumab drug product is a sterile lyophilized solid formulation provided in a single vial, where each vial nominally contains 300 mg of vedolizumab antibody. Reconstituted vedolizumab IV drug product contains 60 mg/mL of active vedolizumab antibody, 50 mM histidine/histidine HCl, 125 mM arginine HCl, 100 mg/mL sucrose, and 0.6 mg/mL polysorbate 80, with a pH of 6.3. Each vial will be reconstituted with 4.8 mL of sterile water for injection. For the 300 mg dose, 5.0 mL will be removed from each vial and diluted into 0.9% sodium chloride to an approximate volume of 250 mL. For the 600 mg dose, 5.0 mL will be removed from each of 2 vials and diluted into 0.9% sodium chloride to an approximate volume of 250 mL. All participants will be infused intravenously at the same time each day throughout the study. Participants will discontinue treatment if they have an unacceptable vedolizumab-related toxicity.

### B. Overview of Study

The study is designed to evaluate the safety, tolerability, and clinical activity of vedolizumab to treat patients who have developed acute intestinal GvHD that is refractory to primary steroid therapy. Clinical GvHD scoring will be used for assessment of response to treatment (Martin P J et al., Biol Blood Marrow Transplant 2009; 15(7):777-84.). Patients with acute intestinal GvHD who have received no systemic therapy for the treatment of acute GvHD (prophylaxis acceptable) other than corticosteroids will be eligible to enroll in the study.

Eligibility will be determined during a screening period, which may last for up to 28 days before Day 1 (designation of the day of the first IV infusion of vedolizumab). Patients who meet all eligibility criteria will be enrolled in this study. Approximately 38 evaluable patients will be enrolled.

Patients will be randomized at a ratio of 1:1 to 2 treatment arms to receive either 300 mg or 600 mg vedolizumab IV on Days 1, 15, 43, 71, and 99. After approximately 10 patients are enrolled at each dose level and have data available from their Day 28 evaluation, safety, tolerability, efficacy, and PK results will be assessed from the patients at both vedolizumab dose levels (300 mg and 600 mg), and a Bayesian statistical approach will be used to facilitate the determination of an appropriate dose for subsequent patients in the study. The cohort at the chosen dose level will then be expanded by approximately 18 additional evaluable patients to further assess the tolerability and effectiveness of vedolizumab. Both dose levels may be expanded based on accumulating results. Patients who respond to and tolerate all 5 planned doses of vedolizumab and who develop recurrent symptoms of intestinal GvHD following discontinuation of therapy (i.e., after the fifth dose) may be eligible to enter an extension phase where they may receive 300 mg vedolizumab IV every 2 weeks for 2 doses followed by Q4W for up to 1 year from the first dose of study drug.

Vital signs, physical and neurological examinations, AE assessments, and laboratory values (chemistry, hematology, and urinalysis) will be obtained to evaluate the safety and tolerability of vedolizumab IV. Vital signs will be obtained during the screening period, and on study days 1, 7, 15, 22, 28, 36, 43, 71, 99, at 4 month follow up, 5 month follow up, 6 month follow up, 9 month follow up and 12 month follow up, and will also be obtained at any dose extension visits. Physical and neurological examinations will be obtained during the screening period, and symptom-directed physical exam will be obtained on study days 1, 7, 15, 22, 28, 36, 43, 71, 99, at 12 month follow up, and will also be obtained at any dose extension visits. Optional endoscopy will be performed to evaluate clinical response to vedolizumab treatment.

Serial blood samples for the evaluation of the PK of vedolizumab will be obtained at study days 1, 2, 3, 5, 7, 9, 11, 15, 16, 18, 20, 22, 24, 28, 32, 36, 40, 43, 71 and 99. Serial blood samples will also be obtained for determination of the serum concentration of anti-vedolizumab antibodies and serum biomarkers (including, but not limited to, IL-6, IL-17, and ST2) (McDonald G B et al., Blood 2015; 126(1):113-20; Ponce D M et al., Biol Blood Marrow Transplant 2015; 21(11) 1985-93.] and/or cellular biomarkers (including, but not limited to, CD8+, CD38+, and CD8+ bright effector memory T cells and CD4+ memory T cells) (Khandelwal P et al., Biol Blood Marrow Transplant 2015; 21(7):1215-22.) that might be correlated with the severity of acute GvHD. Other biomarkers for GvHD (Levine J E et al., Lancet Haematol 2015; 2(1):e21-e9.) that may be tested include citrulline (Vokurka S et al., Med Sci Monit 2013; 19:81-5.), serum intestinal fatty acid binding protein (Van den Abbeele P. et al., ISME J 2013; 7(5):949-61.), and surrogate markers for global intestinal damage (e.g., REG3a (Levine J E et al., Biol Blood Marrow Transplant 2012; 18(1 Suppl):5116-24.) and urine indoxyl sulfate (Weber D. et al., Blood 2015; 126(14):1723-8.). Fecal samples will be collected for analysis of the microbiome at study days 36, 43, 71 and 99.

Changes in health-related quality of life will be assessed using the EQ-5D and FACT-BMT questionnaires. Healthcare resource utilization measures will be collected throughout the study. Toxicity will be evaluated according to National Cancer Institute Common Terminology Criteria for Adverse Events (NCI CTCAE), version 4.03, effective date 14 Jun. 2010 (Common Terminology Criteria for Adverse Events (CTCAE). National Cancer Institute, National Institutes of Health, U.S. Department of Health and Human Services Series v4.03. Jun. 14, 2010. Publication No. 09-5410.).

### C. Duration of Treatment

Patients will receive up to 5 doses of vedolizumab IV (a single dose on each of Days 1, 15, 43, 71, and 99). Upon review and agreement by the medical monitor, patients who respond to and tolerate all 5 planned doses of vedolizumab and who develop recurrent symptoms of intestinal GvHD following discontinuation of therapy (i.e., after the fifth dose) may receive 300 mg vedolizumab IV every 2 weeks for 2 doses followed by every 4 weeks for up to 1 year from the first dose of study drug. A dose other than 300 mg and/or a frequency of administration other than every 4 weeks may be chosen based on accumulating safety, efficacy, and PK results. Patients may receive drug beyond 1 year with the agreement of the investigator and the sponsor if, in the opinion of the investigator, the patient is benefiting from treatment.

### D. Period of Evaluation

Patients may receive vedolizumab unless they experience relapse of the underlying malignancy. Patients will discontinue treatment if they have an unacceptable vedolizumab-related toxicity. All patients will be followed for overall survival (OS) every 3 months until death, withdrawal of consent, termination of the study by the sponsor, or for a maximum of 1 year after the last patient is enrolled in the study. Additionally, patients will be required to participate in an LTFU safety survey 6 months after the last dose of study drug.

### E. Inclusion and Exclusion Criteria

The main criteria for inclusion are: adult patients aged ≥18 years who have received 1 allo-HSCT and have primary steroid-refractory acute GvHD with intestinal disease involvement with a severity index of B, C, or D using the Blood and Marrow Transplant Clinical Trials Network (BMT CTN)-modified International Bone Marrow Transplant Registry Database (IBMTR) index will be enrolled. Patients should have evidence of myeloid engraftment, an Eastern Cooperative Oncology Group performance status of 0 to 3, and an estimated creatinine clearance based on the Cockcroft-Gault estimate of ≥60 mL/minute/1.73 m².

Patients who have chronic GvHD, have relapse of underlying malignancy after allo-HSCT, or have received systemic agents other than corticosteroids for treatment of acute GvHD (other than GvHD prophylaxis agents) will be excluded from the study. Patients with active CNS disease, active cytomegalovirus colitis, or signs and symptoms of PML or any history of PML will also be excluded. In addition, patients with severe hepatic veno-occlusive disease/sinusoidal obstruction syndrome will be excluded. Patients who meet the following criteria are eligible to enroll in the study:

1. Male or female patients aged 18 years or older.

2. Recipient of 1 allo-HSCT but not more than 1 allo-HSCT.

3. Patients with primary steroid-refractory GvHD. Steroid-refractory disease is defined as worsening or no improvement in 5 to 7 days of treatment with methylprednisolone 2 mg/kg or equivalent or lack of a CR after 14 days of primary treatment with methylprednisolone 2 mg/kg or equivalent. Note that patients who develop intestinal GvHD while receiving systemic therapy for other GvHD are still eligible after 5 to 7 days, even if the intestinal GvHD has not been present for the entire duration. Patients who may have received an increase in their steroid dose treatment (e.g., increased methylprednisolone from 1 mg/kg to 2 mg/kg) before enrollment will be eligible, provided the patient has met the definition of steroid refractory above.

4. Eastern Cooperative Oncology Group (ECOG) performance status of 0 to 3 (see, Table 4).

TABLE 4
Eastern Cooperative Oncology Group (ECOG) Scale for Performance Status

| Grade | Description |
|---|---|
| 0 | Normal activity. Fully active, able to carry on all predisease performance |
| | without restriction. |
| 1 | Symptoms but ambulatory. Restricted in physically strenuous activity, but |
| | ambulatory and able to carry out work of a light or sedentary nature (e.g., |
| | light housework, office work). |
| 2 | In bed <50% of the time. Ambulatory and capable of all self-care, but |
| | unable to carry out any work activities. Up and about more than 50% of |
| | waking hours. |
| 3 | In bed >50% of the time. Capable of only limited self-care, confined to bed |
| | or chair more than 50% of waking hours. |
| 4 | 100% bedridden. Completely disabled. Cannot carry on any self-care. |
| | Totally confined to bed or chair. |
| 5 | Dead |

5. Acute GvHD with intestinal disease involvement with a severity index of B, C, or D using the Blood and Marrow Transplant Clinical Trials Network (BMT CTN)-modified International Bone Marrow Transplant Registry Database (IBMTR) index (See Table 1 and Table 3). Note that other organ involvement from acute GvHD is also allowed.

6. Evidence of myeloid engraftment defined by absolute neutrophil count ≥0.5×10⁹/L on 3 consecutive days.

7. Creatinine clearance based on the Cockcroft-Gault estimate of ≥60 mL/minute/1.73 m² for patients with serum creatinine concentrations above institutional limits.

8. Sufficient cognitive ability to reliably complete the RAMP questionnaire at Baseline.

9. Female patients who: are postmenopausal for at least 1 year before the Screening visit, OR Are surgically sterile, OR If they are of childbearing potential, agree to practice one highly effective method of contraception and one additional effective (barrier) method at the same time, from the time of signing the informed consent through 18 weeks after the last dose of study drug, or Agree to practice true abstinence, when this is in line with the preferred and usual life style of the subject. (Periodic abstinence [e.g., calendar, ovulation, symptothermal, and postovulation methods], withdrawal, spermicides only, and lactational amenorrhea are not acceptable methods of contraception. Female and male condoms should not be used together.)

Male patients, even if surgically sterilized (i.e., status post vasectomy), who: Agree to practice effective barrier contraception during the entire study treatment period and through 18 weeks after the last dose of study drug, or Agree to practice true abstinence, when this is in line with the preferred and usual lifestyle of the subject. (Periodic abstinence [e.g., calendar, ovulation, symptothermal, and postovulation methods], withdrawal, spermicides only, and lactational amenorrhea are not acceptable methods of contraception. Female and male condoms should not be used together.)

10. Voluntary written consent must be given before performance of any study-related procedure not part of standard medical care, with the understanding that consent may be withdrawn by the patient at any time without prejudice to future medical care.

11. Suitable venous access for the study-required blood sampling, including PK and biomarker sampling. Patients with a planned central venous access device will be allowed.

Patients meeting any of the following exclusion criteria will not to be enrolled in the study:

1. Presence of chronic GvHD at Screening (including acute-chronic overlap syndrome).

2. Relapsed disease after allo-HSCT.

3. Patients with hyperacute GvHD defined as onset of GvHD within the first 15 days following hematopoietic stem cell infusion.

4. Received systemic agents other than corticosteroids for treatment of acute GvHD. GvHD prophylaxis agents (e.g., calcineurin inhibitors) may be continued.

5. Acute steroid-resistant GvHD beyond 28 days from primary treatment.

6. Patients with a positive PML subjective checklist must be evaluated by a neurologist for possible PML before enrollment (see Section 10.7). Patients will be excluded if PML cannot be ruled out.

7. Evidence of encephalopathy at Screening.

8. Evidence of severe hepatic veno-occlusive disease/sinusoidal obstruction syndrome.

9. Life expectancy of <3 weeks.

10. History of any major neurological disorders, including multiple sclerosis or neurodegenerative disease. Patients with a history of stroke or brain tumor within the past 3 years are also excluded.

11. Patients with active cytomegalovirus (CMV) colitis (see Section 8.5.3).

12. The patient has chronic hepatitis B (HBV) or hepatitis C (HCV) infection indicated by testing for positive HBV surface antigen, and/or HCV RNA.

13. Any identified congenital or acquired immunodeficiency (e.g., common variable immunodeficiency, human immunodeficiency virus [HIV] infection, organ transplantation).

14. Positive Clostridium difficile toxin test on a stool sample or evidence of other intestinal pathogens (e.g., adenovirus) during Screening.

15. Evidence of uncontrolled active systemic infection.

16. Any serious medical or psychiatric condition that could, in the investigator's or medical monitor's opinion, potentially interfere with the completion of treatment according to this protocol.

17. Any unstable or uncontrolled cardiovascular, pulmonary, hepatic, renal, GI, genitourinary, hematological, coagulation, immunological, endocrine/metabolic, neurologic, or other medical disorder that, in the opinion of the investigator or medical monitor, would confound the study results or compromise patient safety.

18. History of hypersensitivity or allergies to vedolizumab or its components.

19. If female, the patient is pregnant or lactating or intending to become pregnant before, during, or within 18 weeks after participating in this study; or intending to donate ova during such time period.

20. If male, the patient intends to donate sperm during the course of this study or for 18 weeks thereafter.

### F. Study Endpoints

The primary and secondary endpoints of the study will be:

### Primary Endpoints and Measures

1. The proportion of subjects with overall response (partial response (PR)+very good partial response (VGPR)+compete response (CR)) at Day 28.

Complete Response (CR) is defined as the resolution of all signs and symptoms of acute graft-versus-host-disease (GvHD).

Very good partial response (VGPR) is defined as resolution of the signs and symptoms of the GvHD: 1) Skin: No rash, or residual erythematous rash involving <25% of the body surface, without bullae (excluding residual faint erythema and hyperpigmentation). 2) Liver: Total serum bilirubin concentration <2 mg/dL or <25% of baseline at enrollment. 3) Gut: a) Participant tolerates food or enteral feeding; b) Predominantly formed stools; c) No overt gastrointestinal bleeding or abdominal cramping; d) No more than occasional nausea or vomiting.

Partial Response (PR) is defined as improvement of 1 GvHD stage in 1 or more organs without progression in any organ.

2. The number and percentage of patients who experience serious adverse events (SAEs) from administration of the first dose of vedolizumab IV through Day 28. An Adverse Event (AE) is defined as any untoward medical occurrence in a clinical investigation participant administered a drug; it does not necessarily have to have a causal relationship with this treatment. An SAE is defined as an untoward medical occurrence, significant hazard, contraindication, side effect or precaution that at any dose: results in death, is life-threatening, required in-patient hospitalization or prolongation of existing hospitalization, results in persistent or significant disability/incapacity, is a congenital anomaly/birth defect or is medically significant. Among these, events which are considered possibly associated with a medicinal product are defined as adverse drug reactions.

Secondary Endpoints

The proportion of subjects who die in the absence of primary malignancy relapse after allo-HSCT at 6 months.

The proportion of subjects with CR at Day 28.

The proportion of subjects with intestinal overall response at Day 28. Overall intestinal response is defined as the resolution of all signs and symptoms of acute intestinal GvHD. Symptoms of acute intestinal GvHD will be measured using Glucksberg and Blood and Marrow Transplant Clinical Trials Network (BMT CTN)-modified criteria for International Bone Marrow Transplant Registry Database (IBMTR) which is a graded scale from 1 to 4, with 1 being the least severe.

OS at 6 and 12 months. OS is defined as the time from the date of enrollment to the date of death, due to any cause.

The proportion of subjects alive without GvHD or relapse of primary malignancy at 6 and 12 months.

The number and percentage of patients who experience treatment emergent adverse events (TEAEs) from administration of the first dose of vedolizumab IV through 18 weeks after administration of the last dose of vedolizumab IV. A TEAE is defined as an adverse event with an onset that occurs after receiving study drug.

The number and percentage of patients who experience SAEs from administration of the first dose of vedolizumab IV through 18 weeks after administration of the last dose of vedolizumab IV.

Mean serum concentrations of vedolizumab before dosing (Ctrough) on Day 99. The total dose of steroids administered (mg/kg/day of methylprednisolone or equivalent) from the start of the first IV infusion of vedolizumab through both 6 and 12 months.

Investigational Endpoints

The proportion of subjects with CR at Days 15, 43, 71, and 99 and at 6 months.

The proportion of subjects with intestinal overall response at Days 15, 43, 71, and 99 and at 6 months.

The proportion of subjects without active GvHD relapse or death at 6 and 12 months.

The proportion of subjects with endoscopic response (optional).

The percentage of patients who develop chronic GvHD requiring systemic immunosuppression.

The presence of anti-vedolizumab antibodies (assessments performed on specimens collected at Baseline and at the end of the exposure period).

The proportion of patients positive for anti-vedolizumab antibody at Baseline, Day 20, and at 6 months.

Changes in serum biomarkers (including, but not limited to, interleukin [IL]-6, IL-17, and suppressor of tumorigenicity 2 [ST2]) and/or cellular biomarkers (including, but not limited to, CD8+, CD38+, and CD8+ bright effector memory T cells and CD4+ memory T cells) that might be correlated with the severity of acute GvHD. Other biomarkers for GvHD that may be tested include citrulline, serum intestinal fatty acid binding protein, and surrogate markers for global intestinal damage (e.g., REG3a and urine indoxyl sulfate).

Changes in the fecal microbiome.

Healthcare resource utilization measures such as: length of hospital stay in days, hospital admission type (intensive care, general ward, emergency), outpatient hospital visits, medications administered during hospital/clinic visits, medical investigations during hospital/clinic visits, and surgical procedures during the length of the study.

Change from baseline in European Quality of Life 5-Dimensional (EQ-5D) scores (Stark R G et al., Infamm Bowel Dis 2010; 16(1):42-51).

Change from baseline in Functional Assessment of Cancer Therapy-Bone Marrow Transplant Scale (FACT-BMT) scores (Parikh A et al., Inflamm Bowel Dis 2012; 18(8)1470-9).

### Example 3

Monte Carlo simulations were run with a population pharmacokinetic model of vedolizumab serum concentration in clinical studies. Simulations included interindividual and residual variability in addition to weight and albumin effects. All other covariates were set to their reference values. One thousand adult patients were simulated in this study. Albumin and weight were randomly sampled from a normal distribution. The simulated dosing regimen was 75 mg of vedolizumab via a 30 minute IV infusion on days −1, +13, +42 (i.e., days 0, 14 and 43 relative to first dose).

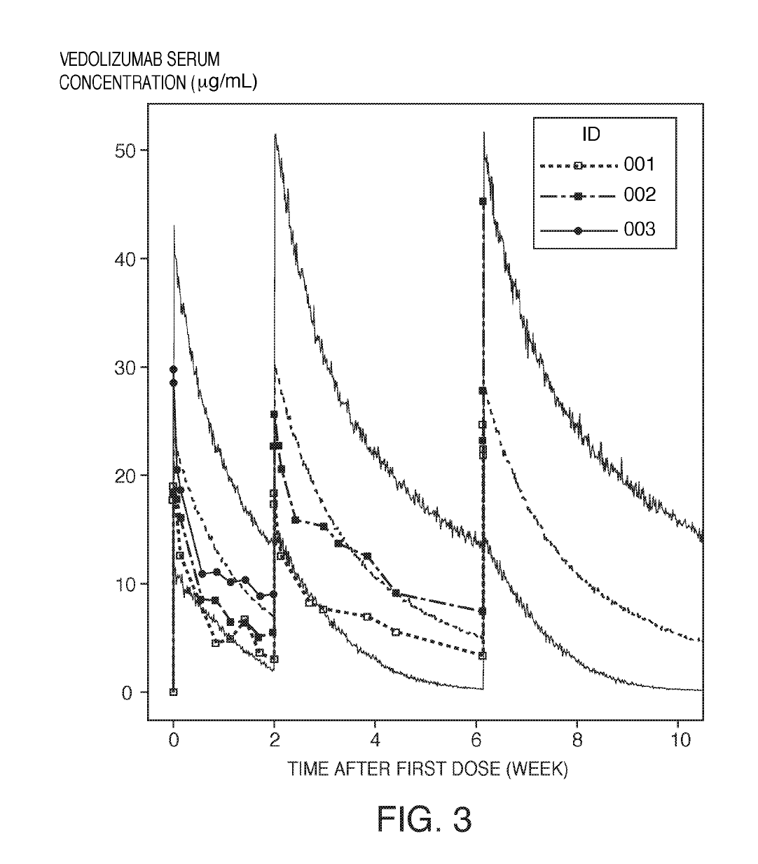

Observed data from three patients enrolled in the phase 1b, open-label, dose-finding study (Example 1) was overlaid with the simulation data (see FIG. 3). The “fuzziness” of the region between the jagged lines is due to residual variability. FIG. 3 illustrates the measured and simulated vedolizumab serum concentration over time. In this figure, the vedolizumab concentration in one patient did not reach 10 μg/ml except immediately after dosing. Another patient retained more than 10 μg/ml vedolizumab for several days after the second dose, but not the first dose. A third patient retained more than 10 μg/ml vedolizumab for several days after the first dose.

```
SEQUENCE DISCLOSURE
SEQ ID NO: 1
Met Gly Trp Ser Cys Ile Ile Leu Phe Leu Val Ala Thr Ala Thr Gly
1 5 10 15
Val His Ser Gln Val Gln Leu Val Gln Ser Gly Ala Glu Val Lys Lys
                        20 25 30
Pro Gly Ala Ser Val Lys Val Ser Cys Lys Gly Ser Gly Tyr Thr Phe
                35 40 45
Thr Ser Tyr Trp Met His Trp Val Arg Gln Ala Pro Gly Gln Arg Leu
        50 55 60
Glu Trp Ile Gly Glu Ile Asp Pro Ser Glu Ser Asn Thr Asn Tyr Asn
65 70 75 80
Gln Lys Phe Lys Gly Arg Val Thr Leu Thr Val Asp Ile Ser Ala Ser
                                85 90 95
Thr Ala Tyr Met Glu Leu Ser Ser Leu Arg Ser Glu Asp Thr Ala Val
                        100 105 110
Tyr Tyr Cys Ala Arg Gly Gly Tyr Asp Gly Trp Asp Tyr Ala Ile Asp
                115 120 125
Tyr Trp Gly Gln Gly Thr Leu Val Thr Val Ser Ser Ala Ser Thr Lys
        130 135 140
Gly Pro Ser Val Phe Pro Leu Ala Pro Ser Ser Lys Ser Thr Ser Gly
145 150 155 160
Gly Thr Ala Ala Leu Gly Cys Leu Val Lys Asp Tyr Phe Pro Glu Pro
                                165 170 175
Val Thr Val Ser Trp Asn Ser Gly Ala Leu Thr Ser Gly Val His Thr
                        180 185 190
Phe Pro Ala Val Leu Gln Ser Ser Gly Leu Tyr Ser Leu Ser Ser Val
                195 200 205
Val Thr Val Pro Ser Ser Ser Leu Gly Thr Gln Thr Tyr Ile Cys Asn
        210 215 220
Val Asn His Lys Pro Ser Asn Thr Lys Val Asp Lys Lys Val Glu Pro
225 230 235 240
Lys Ser Cys Asp Lys Thr His Thr Cys Pro Pro Cys Pro Ala Pro Glu
                                245 250 255
Leu Ala Gly Ala Pro Ser Val Phe Leu Phe Pro Pro Lys Pro Lys Asp
                        260 265 270
Thr Leu Met Ile Ser Arg Thr Pro Glu Val Thr Cys Val Val Val Asp
                275 280 285
Val Ser His Glu Asp Pro Glu Val Lys Phe Asn Trp Tyr Val Asp Gly
        290 295 300
Val Glu Val His Asn Ala Lys Thr Lys Pro Arg Glu Glu Gln Tyr Asn
305 310 315 320
Ser Thr Tyr Arg Val Val Ser Val Leu Thr Val Leu His Gln Asp Trp
                                325 330 335
Leu Asn Gly Lys Glu Tyr Lys Cys Lys Val Ser Asn Lys Ala Leu Pro
                        340 345 350
Ala Pro Ile Glu Lys Thr Ile Ser Lys Ala Lys Gly Gln Pro Arg Glu
                355 360 365
Pro Gln Val Tyr Thr Leu Pro Pro Ser Arg Asp Glu Leu Thr Lys Asn
        370 375 380
Gln Val Ser Leu Thr Cys Leu Val Lys Gly Phe Tyr Pro Ser Asp Ile
385 390 395 400
Ala Val Glu Trp Glu Ser Asn Gly Gln Pro Glu Asn Asn Tyr Lys Thr
                                405 410 415
Thr Pro Pro Val Leu Asp Ser Asp Gly Ser Phe Phe Leu Tyr Ser Lys
                        420 425 430
Leu Thr Val Asp Lys Ser Arg Trp Gln Gln Gly Asn Val Phe Ser Cys
                435 440 445
Ser Val Met His Glu Ala Leu His Asn His Tyr Thr Gln Lys Ser Leu
        450 455 460
Ser Leu Ser Pro Gly Lys
465 470
SEQ ID NO: 2
Met Gly Trp Ser Cys Ile Ile Leu Phe Leu Val Ala Thr Ala Thr Gly
1 5 10 15
Val His Ser Asp Val Val Met Thr Gln Ser Pro Leu Ser Leu Pro Val
                        20 25 30
Thr Pro Gly Glu Pro Ala Ser Ile Ser Cys Arg Ser Ser Gln Ser Leu
                35 40 45
Ala Lys Ser Tyr Gly Asn Thr Tyr Leu Ser Trp Tyr Leu Gln Lys Pro
        50 55 60
Gly Gln Ser Pro Gln Leu Leu Ile Tyr Gly Ile Ser Asn Arg Phe Ser
65 70 75 80
Gly Val Pro Asp Arg Phe Ser Gly Ser Gly Ser Gly Thr Asp Phe Thr
                                85 90 95
Leu Lys Ile Ser Arg Val Glu Ala Glu Asp Val Gly Val Tyr Tyr Cys
                        100 105 110
Leu Gln Gly Thr His Gln Pro Tyr Thr Phe Gly Gln Gly Thr Lys Val
                115 120 125
Glu Ile Lys Arg Thr Val Ala Ala Pro Ser Val Phe Ile Phe Pro Pro
        130 135 140
Ser Asp Glu Gln Leu Lys Ser Gly Thr Ala Ser Val Val Cys Leu Leu
145 150 155 160
Asn Asn Phe Tyr Pro Arg Glu Ala Lys Val Gln Trp Lys Val Asp Asn
                                165 170 175
Ala Leu Gln Ser Gly Asn Ser Gln Glu Ser Val Thr Glu Gln Asp Ser
                        180 185 190
Lys Asp Ser Thr Tyr Ser Leu Ser Ser Thr Leu Thr Leu Ser Lys Ala
                195 200 205
Asp Tyr Glu Lys His Lys Val Tyr Ala Cys Glu Val Thr His Gln Gly
        210 215 220
Leu Ser Ser Pro Val Thr Lys Ser Phe Asn Arg Gly Glu Cys
225 230 235
SEQ ID NO: 3
Asp Val Val Met Thr Gln Ser Pro Leu Ser Leu Pro Val Thr Pro Gly
1 5 10 15
Glu Pro Ala Ser Ile Ser Cys Arg Ser Ser Gln Ser Leu Ala Lys Ser
                        20 25 30
Tyr Gly Asn Thr Tyr Leu Ser Trp Tyr Leu Gln Lys Pro Gly Gln Ser
                35 40 45
Pro Gln Leu Leu Ile Tyr Gly Ile Ser Asn Arg Phe Ser Gly Val Pro
        50 55 60
Asp Arg Phe Ser Gly Ser Gly Ser Gly Thr Asp Phe Thr Leu Lys Ile
65 70 75 80
Ser Arg Val Glu Ala Glu Asp Val Gly Val Tyr Tyr Cys Leu Gln Gly
                                85 90 95
Thr His Gln Pro Tyr Thr Phe Gly Gln Gly Thr Lys Val Glu Ile Lys
                        100 105 110
Arg Ala Asp Ala Ala Pro Ser Val Phe Ile Phe Pro Pro Ser Asp Glu
                115 120 125
Gln Leu Lys Ser Gly Thr Ala Ser Val Val Cys Leu Leu Asn Asn Phe
        130 135 140
Tyr Pro Arg Glu Ala Lys Val Gln Trp Lys Val Asp Asn Ala Leu Gln
145 150 155 160
Ser Gly Asn Ser Gln Glu Ser Val Thr Glu Gln Asp Ser Lys Asp Ser
                                165 170 175
Thr Tyr Ser Leu Ser Ser Thr Leu Thr Leu Ser Lys Ala Asp Tyr Glu
                        180 185 190
Lys His Lys Val Tyr Ala Cys Glu Val Thr His Gln Gly Leu Ser Ser
                195 200 205
Pro Val Thr Lys Ser Phe Asn Arg Gly Glu Cys
        210 215
SEQ ID NO: 4
Ser Tyr Trp Met His
1 5
SEQ ID NO: 5
Glu Ile Asp Pro Ser Glu Ser Asn Thr Asn Tyr Asn Gln Lys Phe Lys
1 5 10 15
Gly
SEQ ID NO: 6
Gly Gly Tyr Asp Gly Trp Asp Tyr Ala Ile Asp Tyr
1 5 10
SEQ ID NO: 7
Arg Ser Ser Gln Ser Leu Ala Lys Ser Tyr Gly Asn Thr Tyr Leu Ser
1 5 10 15
SEQ ID NO: 8
Gly Ile Ser Asn Arg Phe Ser
1 5
SEQ ID NO: 9
Leu Gln Gly Thr His Gln Pro Tyr Thr
1 5
SEQ ID NO: 10
Asp Ile Val Met Thr Gln Ser Pro Leu Ser Leu Pro Val Thr Pro Gly
1 5 10 15
Glu Pro Ala Ser Ile Ser Cys Arg Ser Ser Gln Ser Leu Leu His Ser
                        20 25 30
Asn Gly Tyr Asn Tyr Leu Asp Trp Tyr Leu Gln Lys Pro Gly Gln Ser
                35 40 45
Pro Gln Leu Leu Ile Tyr Leu Gly Ser Asn Arg Ala Ser Gly Val Pro
        50 55 60
Asp Arg Phe Ser Gly Ser Gly Ser Gly Thr Asp Phe Thr Leu Lys Ile
65 70 75 80
Ser Arg Val Glu Ala Glu Asp Val Gly Val Tyr Tyr Cys Met Gln Ala
                                85 90 95
Leu Gln Thr Pro Gln Thr Phe Gly Gln Gly Lys Val Glu Ile Lys
                        100 105 110
SEQ ID NO: 11
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Ser Tyr
                        20 25 30
Ala Met His Trp Val Arg Gln Ala Pro Gly Gln Arg Leu Glu Trp Met
                35 40 45
Gly Trp Ile Asn Ala Gly Asn Gly Asn Thr Lys Tyr Ser Gln Lys Phe
        50 55 60
Gln Gly Arg Val Thr Ile Thr Arg Asp Thr Ser Ala Ser Thr Ala Tyr
65 70 75 80
Met Glu Leu Ser Ser Leu Arg Ser Glu Asp Thr Ala Val Tyr Tyr Cys
                                85 90 95
Ala Arg Gly Gly Tyr Tyr Gly Ser Gly Ser Asn Tyr Trp Gly Gln Gly
                        100 105 110
Thr Leu Val Thr Val Ser Ser
                115
```

1. A method for treating graft versus host disease (GvHD) in a human, comprising administering to a human in need thereof an antibody that has binding specificity for the human α4β7 integrin complex, wherein the antibody is administered according to the following regimen: a) a first dose of antibody; b) a second dose of antibody about two weeks after the first dose; c) a third dose of antibody about four weeks after the second dose; and optionally d) further doses of antibody, wherein each further dose is administered about four weeks after the immediate prior dose; and wherein each dose in a)-d) is 300 mg, or each dose in a)-d) is 600 mg.

2. The method of claim 1, wherein the GvHD is acute GvHD.

3. The method of claim 2, wherein the acute GvHD is steroid refractory acute GvHD.

4. The method of any one of claims 1-3, wherein the human has steroid refractory acute GvHD with intestinal disease involvement with a severity index of B, C, or D using the BMT CTN-modified IBMTR index.

5. The method of any one of claims 1-4, wherein the human in need thereof has received an allogenic hematopoietic stem cell transplant.

6. The method of claim 5, wherein the human in need thereof has myeloid engraftment.

7. The method of any one of claims 1-6, wherein the human in need thereof has and Eastern Cooperative Oncology Group (ECOC) performance status of 0 to 3.

8. The method of any one of claims 1-7, wherein the human in need thereof has a creatinine clearance of ≥60 mL/minute/1.73 m², based on the Cockcroft-Gault estimate.

9. The method of any one of claims 1-8, wherein the antibody is administered intravenously.

10. The method of claim 9, wherein the antibody is administered as an infusion.

11. The method of claim 10, wherein the antibody is infused over a period of about 30 to about 60 minutes.

12. The method of any one of claims 1-11, wherein the antibody comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

13. The method of any one of claims 1-12, wherein the antibody has a heavy chain variable region sequence of amino acids 20 to 140 of SEQ ID NO:1.

14. The method of any one of claims 1-12, wherein the antibody has a light chain variable region sequence of amino acids 20 to 131 of SEQ ID NO:2.

15. The method of claim 1-12, wherein the antibody has a heavy chain comprising amino acids 20 to 470 of SEQ ID NO:1 and a light chain comprising amino acids 20 to 238 of SEQ ID NO:2.

16. The method of any one of claims 1-15, wherein the antibody is a humanized antibody.

17. The method of claim 16, wherein the antibody is vedolizumab.

18. A method of reducing the severity of acute graft versus host disease (GvHD), wherein the method comprises the step of:
administering to a human patient undergoing allogeneic hematopoietic stem cell transplantation (allo-HSCT), wherein the patient is at risk of acute GvHD, a humanized antibody having binding specificity for human α4β7 integrin,
wherein the humanized antibody is administered to the patient according to the following dosing regimen:
a. an initial dose of 300 mg, 450 mg or 600 mg of the humanized antibody as an intravenous infusion after allo-HSCT;
b. followed by a second subsequent dose of 300 mg of the humanized antibody as an intravenous infusion at about two weeks after the initial dose;
c. followed by a third subsequent dose of 300 mg of the humanized antibody as an intravenous infusion at about six weeks after the initial dose;
wherein the humanized antibody comprises an antigen binding region of nonhuman origin and at least a portion of an antibody of human origin, wherein the humanized antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6,
thereby reducing the occurrence of GvHD.

19. The method of claim 18, wherein reducing the severity of acute graft versus host disease (GvHD) results in Grade I or Grade II GvHD, per modified Glucksberg criteria, or similar severity of GvHD per other scoring system, or no GvHD.

20. The method of claim 18, wherein reducing the severity of acute GvHD is a 50% reduction in cumulative incidence and severity of Grade II-IV or Grade III-IV acute GvHD at Day 100 as compared to treatment with methotrexate and calcineurin inhibitor alone.

21. The method of claim 18, wherein reducing the severity of acute graft versus host disease (GvHD) is a reduction in 1 year mortality as compared to treatment with methotrexate and calcineurin inhibitor alone.

22. The method of claim 18, wherein the patient is identified as at risk of acute GvHD after measurement of criteria selected from the group consisting of biomarkers, clinical signs and refractoriness to steroid use.

23. The method of claim 18, wherein the humanized antibody is administered more than 15 days after hematopoietic stem cell infusion.

24. A method of suppressing an immune response in a cancer patient, wherein the method comprises the step of:
administering to a human patient undergoing allogeneic hematopoietic stem cell transplantation (allo-HSCT), a humanized antibody having binding specificity for human α4β7 integrin,
wherein the humanized antibody is administered to the patient according to the following dosing regimen:
a. an initial dose of 300 mg, 450 mg or 600 mg of the humanized antibody as an intravenous infusion the day before allo-HSCT;
b. followed by a second subsequent dose of 300 mg of the humanized antibody as an intravenous infusion at about two weeks after the initial dose;
c. followed by a third subsequent dose of 300 mg of the humanized antibody as an intravenous infusion at about six weeks after the initial dose;
further wherein the humanized antibody comprises an antigen binding region of nonhuman origin and at least a portion of an antibody of human origin, wherein the humanized antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

25. A method of treating a transplant patient, wherein the transplant patient is a recipient of an infusion of allogeneic hematopoietic cells, comprising administering an anti-α4β7 antagonist.

26. The method of claim 25, wherein, prior to the infusion, the transplant patient is the recipient of conditioning therapy selected from myeloablative conditioning or reduced intensity conditioning.

27. The method of claim 25 or 26, wherein the anti-α4β7 antagonist is administered prior to the infusion.

28. The method of claim 25 or 26, wherein the anti-α4β7 antagonist is administered in multiple doses, with at least one dose prior to the infusion.

29. The method of claim 25 or 26, wherein the anti-α4β7 antagonist is administered in multiple doses, with the first dose on the same day as the infusion.

30. The method of claim 25 or 26, wherein the anti-α4β7 antagonist is administered in multiple doses, with the first dose on the next day after the infusion.

31. The method of claim 25 or 26, wherein the anti-α4β7 antagonist is administered as a single dose 10 to 28 days after the infusion.

32. The method of claim 27 or 28, wherein a dose of anti-α4β7 antagonist is administered between conditioning and the infusion.

33. The method of anyone of claims 25 to 32, wherein the transplant patient is suffering from cancer.

34. The method of claim 33, wherein the cancer is a hematological cancer.

35. The method of claim 34, wherein the hematological cancer is leukemia, lymphoma, myeloma or a myeloproliferative neoplasm.

36. The method of claim 35, wherein the leukemia is acute lymphoblastic leukemia (ALL) or acute myeloid leukemia (AML).

37. The method of any one of claims 25 to 32, wherein the transplant patient is suffering from a nonmalignant hematological or immune disease.

38. The method of claim 37, wherein the nonmalignant hematological or immune disease is selected from the group consisting of hemoglobinopathy, bone marrow failure syndrome, and immune disease.

39. The method of any one of claims 25 to 38, wherein the anti-α4β7 antagonist is an anti-α4β7 antibody which has binding specificity for the α4β7 integrin complex.

40. The method of claim 39, wherein the anti-α4β7 antibody is a humanized antibody, wherein the antigen-binding region of the humanized antibody comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

41. The method of claim 40, wherein the humanized antibody is reconstituted from a lyophilized formulation.

42. The method of claim 39 or 40, wherein the humanized antibody is administered intravenously.

43. The method of any one of claims 40 to 42, wherein the humanized antibody has a heavy chain variable region sequence of amino acids 20 to 140 of SEQ ID NO:1.

44. The method of any one of claims 40 to 43, wherein the humanized antibody has a light chain variable region sequence of amino acids 20 to 131 of SEQ ID NO:2.

45. The method of claim 43 or 44, wherein the humanized antibody has a heavy chain comprising amino acids 20 to 470 of SEQ ID NO:1 and a light chain comprising amino acids 20 to 238 of SEQ ID NO:2.

46. The method of any one of claims 40 to 45, wherein the humanized antibody is vedolizumab.

47. The method of any one of claims 25 to 46, further comprising treating the transplant patient with tacrolimus, tacrolimus and methotrexate or methotrexate.

48. The method of any one of claims 25 to 47, further comprising detecting engraftment of the allo-HSCs by measuring neutrophil number.

49. The method of claim 48, further comprising measuring a biomarker selected from the group consisting of interleukin-6 (IL-6), interleukin-17 (IL-17), suppressor of tumorigenicity 2 (ST2), CD8+ cells, CD38+ cells, CD8+ bright effector memory T cells, and CD4+ memory T cells, wherein the amount of the biomarker measured before or within one week after the infusion and the amount of the biomarker measured at a time 20 to 100 days after the infusion is unchanged.

50. The method of any one of claims 25 to 49, wherein the patient has an adverse event that does not include stage 3 or stage 4 GvHD of the intestine.

51. The method of any one of claims 25 to 50, wherein the allogeneic hematopoietic cells are allogeneic hematopoietic stem cells.

52. The method of any one of claims 25 to 50, wherein the allogeneic hematopoietic cells are allogeneic leukocytic cells.

53. The method of claim 52, wherein the allogeneic leukocytic cells are T-lymphocytes.

54. A method of preventing graft versus host disease (GvHD), wherein the method comprises the step of:
administering to a human patient undergoing allogeneic hematopoietic stem cell transplantation (allo-HSCT), a humanized antibody having binding specificity for human α4β7 integrin,
wherein the humanized antibody is administered to the patient according to the following dosing regimen:
a. an initial dose of 75 mg, 300 mg, 450 mg or 600 mg of the humanized antibody as an intravenous infusion the day before allo-HSCT;
b. followed by a second subsequent dose of 75 mg, 300 mg, 450 mg or 600 mg of the humanized antibody as an intravenous infusion at about two weeks after the initial dose;
c. followed by a third subsequent dose of 75 mg, 300 mg, 450 mg or 600 mg of the humanized antibody as an intravenous infusion at about six weeks after the initial dose;
further wherein the humanized antibody comprises an antigen binding region of nonhuman origin and at least a portion of an antibody of human origin, wherein the humanized antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

55. The method of claim 54, wherein the dosing regimen results in Grade II GvHD, Grade I GvHD or no GvHD.

56. The method of claim 54, wherein said preventing results in sustained α4β7-blockade at the time of hematopoietic stem cell infusion.

57. The method of claim 54 or 55, wherein tacrolimus is co-administered to the human patient.

58. The method of any one of claims 54 to 57, wherein methotrexate is co-administered to the human patient.

59. The method of any one of claims 54 to 58, wherein the humanized antibody is administered to the patient over about 30 minutes.

60. The method of any one of claims 54 to 59, wherein the humanized antibody is reconstituted from a lyophilized formulation.

61. The method of claim 60, further wherein the humanized antibody is reconstituted to comprise a stable liquid formulation.

62. The method of any one of claims 54 to 61, wherein the humanized antibody has a heavy chain variable region sequence of amino acids 20 to 140 of SEQ ID NO:1.

63. The method of any one of claims 54 to 62, wherein the humanized antibody has a light chain variable region sequence of amino acids 20 to 131 of SEQ ID NO:2.

64. The method of claim 62 or 63, wherein the humanized antibody has a heavy chain comprising amino acids 20 to 470 of SEQ ID NO:1 and a light chain comprising amino acids 20 to 238 of SEQ ID NO:2.

65. The method of any one of claims 54 to 64, wherein the humanized antibody is vedolizumab.

66. A method of treating a patient suffering from cancer or a nonmalignant hematological, immunological disease or autoimmune disease, comprising the steps of:
a. conditioning the immune system of the patient for hematopoietic stem cell transplant,
b. administering a humanized antibody having binding specificity for human α4β7 integrin,
c. waiting at least 12 hours,
d. administering allogeneic hematopoietic stem cells,
e. waiting thirteen days, then administering a second dose of humanized antibody having binding specificity for human α4β7 integrin, and
f. waiting four weeks, then administering a third dose of humanized antibody having binding specificity for human α4β7 integrin.

67. The method of claim 66, further comprising administering tacrolimus to the patient.

68. The method of claim 66 or 67, further comprising administering methotrexate to the patient.

69. The method of any one of claims 66 to 68, wherein the conditioning of the immune system is myeloablative conditioning or reduced intensity conditioning.

70. The method of any one of claims 66 to 69, wherein the patient has an adverse event that does not include stage 3 or stage 4 GvHD of the intestine.

71. The method of any one of claims 66 to 69, wherein the patient has an adverse event that does not include grade III or grade IV GvHD.

72. The method of any one of claims 66 to 69, wherein the patient has leukemia or lymphoma.

73. The method of any one of claims 66 to 69, wherein the allogeneic hematopoietic stem cells are from peripheral blood.

74. The method of any one of claims 66 to 69, wherein the allogeneic hematopoietic stem cells engraft without further immunosuppressive therapy.

75. The method of any one of claims 66 to 69, wherein the humanized antibody comprises an antigen binding region of nonhuman origin and at least a portion of an antibody of human origin, wherein the humanized antibody has binding specificity for the α4β7 complex, wherein the antigen-binding region comprises the CDRs:
Light chain: CDR1 SEQ ID NO:7
CDR2 SEQ ID NO:8 and
CDR3 SEQ ID NO:9; and
Heavy chain: CDR1 SEQ ID NO:4
CDR2 SEQ ID NO:5 and
CDR3 SEQ ID NO:6.

76. The method of claim 75, wherein the humanized antibody is reconstituted from a lyophilized formulation.

77. The method of claim 75, wherein the humanized antibody has a heavy chain variable region sequence of amino acids 20 to 140 of SEQ ID NO:1.

78. The method of claim 75, wherein the humanized antibody has a light chain variable region sequence of amino acids 20 to 131 of SEQ ID NO:2.

79. The method of any one of claims 75 to 78, wherein the humanized antibody has a heavy chain comprising amino acids 20 to 470 of SEQ ID NO:1 and a light chain comprising amino acids 20 to 238 of SEQ ID NO:2.

80. The method of any one of claims 75 to 78, wherein the humanized antibody is vedolizumab.


SEQUENCE LISTING (11 sequences)

SEQ ID NO: 1 - protein, 470 aa, Artificial Sequence
  source: /note=“Description of Artificial Sequence Synthetic polypeptide”
MGWSCIILFLVATATGVHSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, Artificial Sequence
  source: /note=“Description of Artificial Sequence Synthetic polypeptide”
MGWSCIILFLVATATGVHSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 219 aa, Artificial Sequence
  source: /note=“Description of Artificial Sequence Synthetic polypeptide”
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRADAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 5 aa, Artificial Sequence
  source: /note=“Description of Artificial Sequence Synthetic peptide”
SYWMH

SEQ ID NO: 5 - protein, 17 aa, Artificial Sequence
  source: /note=“Description of Artificial Sequence Synthetic peptide”
EIDPSESNTNYNQKFKG

SEQ ID NO: 6 - protein, 12 aa, Artificial Sequence
  source: /note=“Description of Artificial Sequence Synthetic peptide”
GGYDGWDYAIDY

SEQ ID NO: 7 - protein, 16 aa, Artificial Sequence
  source: /note=“Description of Artificial Sequence Synthetic peptide”
RSSQSLAKSYGNTYLS

SEQ ID NO: 8 - protein, 7 aa, Artificial Sequence
  source: /note=“Description of Artificial Sequence Synthetic peptide”
GISNRFS

SEQ ID NO: 9 - protein, 9 aa, Artificial Sequence
  source: /note=“Description of Artificial Sequence Synthetic peptide”
LQGTHQPYT

SEQ ID NO: 10 - protein, 111 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPQTFGQGKVEIK

SEQ ID NO: 11 - protein, 119 aa, Artificial Sequence
  source: /note=“Description of Artificial Sequence Synthetic polypeptide”
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGGYYGSGSNYWGQGTLVTVSS